(19) United States
(12) Patent Application Publication
(10) Pub. No.: US 2024/0382149 A1
(43) Pub. Date: Nov. 21, 2024

(54) DIGITAL IMAGING AND ARTIFICIAL INTELLIGENCE-BASED SYSTEMS AND METHODS FOR ANALYZING PIXEL DATA OF AN IMAGE OF USER SKIN TO GENERATE ONE OR MORE USER-SPECIFIC SKIN SPOT CLASSIFICATIONS

(71) Applicant: The Procter & Gamble Company, Cincinnati, OH (US)

(72) Inventors: Dissanayake Mudiyanselage Mahathma Bandara Dissanayake, Mason, OH (US); Joseph Michael Zukowski, Blue Ash, OH (US); Junko Matsumoto Agerton, Mason, OH (US)

(21) Appl. No.: 18/198,883

(22) Filed: May 18, 2023

(51) Int. Cl. A61B 5/00; G06T 7/00; G06V 10/44; G06V 10/764; G16H 50/20

(52) U.S. Cl. CPC A61B 5/444; A61B 5/443; G06T 7/0012; G06V 10/44; G06V 10/764; G16H 50/20; G06T 2207/20132; G06T 2207/30088

(57) ABSTRACT

Digital imaging and artificial intelligence-based systems and methods are described for analyzing pixel data of an image of user skin to generate one or more user-specific skin spot classifications. A digital image of a user is received at an imaging application (app) and comprises pixel data of at least a portion of a skin region of the user. A skin-based learning model, trained with pixel data of a plurality of training images depicting skin of respective individuals, analyzes the image to determine at least one spot classification of the user's skin. The imaging app generates, based on the at least one spot classification, a user-specific skin recommendation designed to address at least one spot feature identifiable within the pixel data comprising the portion of the skin region of the user.

### FIELD

The present disclosure generally relates to digital imaging and artificial intelligence-based systems and methods, and, more particularly, to digital imaging and artificial intelligence-based systems and methods for analyzing pixel data of an image of user skin to generate one or more user-specific skin spot classifications.

### BACKGROUND

Generally, human skin can experience variations and/or discoloration in given areas, such as the face. The variations and/or discoloration can occur in various aspects in the form of one or more spots on the skin surface. Such spots can be of various types, shapes, and sizes, shades, and/or colors. For example, one type of spot can be a red spot, which can be a hemoglobin related spot. A hemoglobin related spot can occur where blood pools under the skin and leave a residue of hemoglobin that settles in the tissue there. Hemoglobin contains iron, which causes a red or rusty skin color in the effected area. As another example, another type of spot can be a brown spot, which can be melanin related spot. Generally, melanin is a substance that can augment skin pigmentation. Melanin in the skin can depend on different factors, including genetics and sun exposure. Generally, skin spots, and additional and/or different spot types of a person's skin, can be caused by various endogenous and/or exogenous factors.

Such spots can be similar in shape and size and can be difficult to determine what type of spot has formed on the skin, or otherwise what the underlying cause of a given spot is. This can lead to problems involving incorrect identification of such spots. Incorrect identification can, in turn, can lead to ineffective treatment of such spots. For example, a product designed to treat a hemoglobin related spot can be applied to a melanin related spot (or vice versa), which, on the one hand, can at least be ineffective, and on the other hand can be potentially dangerous (e.g., application of a prescription medication for a skin spot that is different than what the medication is intended for). This problem is exacerbated given the complexity of skin types, especially when considered across different users, each of whom may be associated with different demographics, races, and/or ethnicities. This creates a problem in the diagnosis and treatment of various human skin conditions and characteristics. For example, prior art methods, including personal consumer product trials can be time consuming or error prone (and possibly negative). In addition, a user may attempt to empirically experiment with various products or techniques, but without achieving satisfactory results and/or causing possible negative side effects, impacting the health or otherwise visual appearance of his or her skin.

For the foregoing reasons, there is a need for digital imaging and artificial intelligence-based systems and methods for analyzing pixel data of an image of user skin to generate one or more user-specific skin spot classifications.

### SUMMARY

Generally, as described herein, digital imaging and artificial intelligence-based systems are described for analyzing pixel data of a skin region of a user to generate one or more user-specific spot classification(s). Such digital imaging and artificial intelligence-based systems provide a digital imaging, and artificial intelligence (AI), based solution for overcoming problems that arise from the difficulties in identifying and treating various endogenous and/or exogenous factors or attributes affecting the health of human skin.

The digital imaging and artificial intelligence-based systems as described herein allow a user to submit a specific user image to imaging server(s) (e.g., including its one or more processors), or otherwise a computing device (e.g., such as locally on the user's mobile device), where the imaging server(s) or user computing device, implements or executes an artificial intelligence based skin-based learning model trained with pixel data of potentially 10,000s (or more) images depicting skin or skin regions of respective individuals. The skin-based learning model may generate, based on an image classification of the user's skin, at least one user-specific spot classification designed to address at least one feature identifiable within the pixel data comprising the at least the portion of a skin region of the user. For example, an image of the user's skin can comprise pixels or pixel data indicative of spots (e.g., hemoglobin and/or melanin related spots) or other attributes/conditions of a specific user's skin. In some embodiments, the user-specific spot classification (and/or product specific spot classification) may be transmitted via a computer network to a user computing device of the user for rendering on a display screen. In other embodiments, no transmission to the imaging server of the user's specific image occurs, where the user-specific spot classification (and/or product specific spot classification) may instead be generated by the skin-based learning model, executing and/or implemented locally on the user's mobile device and rendered, by a processor of the mobile device, on a display screen of the mobile device. In various embodiments, such rendering may include graphical representations, overlays, annotations, and the like for addressing the feature in the pixel data.

More specifically, as described herein, a digital imaging and artificial intelligence-based system configured to analyze pixel data of an image of user skin to generate one or more user-specific skin spot classifications is disclosed. The digital imaging and artificial intelligence-based system is configured to analyze pixel data of an image of user skin to generate one or more user-specific skin spot classifications. The digital imaging and artificial intelligence-based system may include one or more processors and an imaging application (app) comprising computing instructions configured to execute on the one or more processors. The digital imaging and artificial intelligence-based system may further comprise a skin-based learning model, accessible by the imaging app, and trained with pixel data of a plurality of training images depicting skin of respective individuals. The skin-based learning model may be configured to output one or more spot classifications corresponding to one or more spot features of skin regions of the respective individuals. The computing instructions of the imaging app when executed by the one or more processors may cause the one or more processors to receive an image of a user, where the image comprises a digital image as captured by an imaging device, and the where the image comprise pixel data of at least a portion of a skin region of the user. The computing instructions of the imaging app when executed by the one or more processors may further cause the one or more processors to analyze, by the skin-based learning model, the image as captured by the imaging device to determine at least one spot classification of the user's skin. The at least one spot classification may be selected from the one or more spot classifications of the skin-based learning model. The computing instructions of the imaging app when executed by the one or more processors may further cause the one or more processors to generate, based on the at least one spot classification of the user's skin, a user-specific skin recommendation designed to address at least one spot feature identifiable within the pixel data comprising the portion of the skin region of the user.

In addition, as described herein, a digital imaging and artificial intelligence-based method is disclosed for analyzing pixel data of an image of user skin to generate one or more user-specific skin spot classifications. The digital imaging and artificial intelligence-based method comprises receiving, at one or more processors, an image of a user, the image comprising a digital image as captured by an imaging device, and the image comprising pixel data of at least a portion of a skin region of the user. The digital imaging and artificial intelligence-based method may further comprise analyzing, by a skin-based learning model executing on the one or more processors, the image as captured by the imaging device to determine at least one spot classification of the user's skin. The at least one spot classification may be selected from the one or more spot classifications of the skin-based learning model. In various aspects, the skin-based learning model may be trained with pixel data of a plurality of training images depicting skin of respective individuals, where the skin-based learning model configured to output one or more spot classifications corresponding to one or more spot features of skin regions of the respective individuals. The digital imaging and artificial intelligence-based method may further comprise generating by the one or more processors and based on the at least one spot classification of the user's skin, a user-specific skin recommendation designed to address at least one spot feature identifiable within the pixel data comprising the portion of the skin region of the user.

Still further, as described herein, a tangible, non-transitory computer-readable medium storing instructions for analyzing pixel data of an image of user skin to generate one or more user-specific skin spot classifications is disclosed. The instructions, when executed by one or more processors, may cause the one or more processors to receive an image of a user. The image may comprise a digital image as captured by an imaging device and may comprise pixel data of at least a portion of a skin region of the user. The instructions, when executed by one or more processors, may further cause the one or more processors to analyze, by a skin-based learning model, the image as captured by the imaging device to determine at least one spot classification of the user's skin. The at least one spot classification may be selected from the one or more spot classifications of the skin-based learning model. In various aspects, the skin-based learning model may be trained with pixel data of a plurality of training images depicting skin of respective individuals. The skin-based learning model may be configured to output one or more spot classifications corresponding to one or more spot features of skin regions of the respective individuals. The instructions, when executed by one or more processors, may further cause the one or more processors to generate, based on the at least one spot classification of the user's skin, a user-specific skin recommendation designed to address at least one spot feature identifiable within the pixel data comprising the portion of the skin region of the user.

In accordance with the above, and with the disclosure herein, the present disclosure includes improvements in computer functionality or in improvements to other technologies at least because the disclosure describes that, e.g., an imaging server, or otherwise computing device (e.g., a user computer device), is improved where the intelligence or predictive ability of the server or computing device is enhanced by a trained (e.g., machine learning trained) skin-based learning model. The skin-based learning model, executing on the imaging server or computing device, is able to more accurately identify, based on pixel data of various individuals, one or more of a user-specific skin or spot feature, an image classification of the user's skin region, and/or a user-specific skin recommendation designed to address at least one feature identifiable within the pixel data comprising the at least the portion of a skin region of the user. That is, the present disclosure describes improvements in the functioning of the computer itself or “any other technology or technical field” because an imaging server or user computing device is enhanced with a plurality of training images (e.g., 10,000s of training images and related pixel data as feature data) to accurately predict, detect, classify, or determine pixel data of a user-specific images, such as newly provided user images. This improves over the prior art at least because existing systems lack such predictive or classification functionality and are simply not capable of accurately analyzing user-specific images to output a predictive result to address at least one feature identifiable within the pixel data comprising the at least the portion of a skin region of the a given user.

For similar reasons, the present disclosure relates to improvements to other technologies or technical fields at least because the present disclosure describes or introduces improvements to computing devices in the skin care field and skin care products field, whereby the trained skin-based learning model executing on the imaging device(s) or computing devices improves the field of skin care, chemical formulations and/or skin classifications and identification thereof, with digital and/or artificial intelligence based analysis of user or individual images to output a predictive result to address user-specific pixel data of at least one feature identifiable within the pixel data comprising the at least the portion of a skin region of a given user.

In addition, the present disclosure relates to improvement to other technologies or technical fields at least because the present disclosure describes or introduces improvements to computing devices in the skin care and/or skin care products field, whereby the trained skin-based learning model executing on the imaging device(s) or computing device(s) improve the underlying computer device (e.g., imaging server(s) and/or user computing device), where such computer devices are made more efficient by the configuration, adjustment, or adaptation of a given machine-learning network architecture. For example, in some embodiments, fewer machine resources (e.g., processing cycles or memory storage) may be used by decreasing computational resources by decreasing machine-learning network architecture needed to analyze images, including by reducing depth, width, image size, or other machine-learning based dimensionality requirements. Such reduction frees up the computational resources of an underlying computing system, thereby making it more efficient.

Still further, the present disclosure relates to improvement to other technologies or technical fields at least because the present disclosure describes or introduces improvements to computing devices in the field of security, where images of users are preprocessed (e.g., cropped or otherwise modified) to define extracted or depicted skin regions of a user without depicting personal identifiable information (PII) of the user. For example, simple cropped or redacted portions of an image of a user may be used by the skin-based learning model described herein, which eliminates the need of transmission of private photographs of users across a computer network (where such images may be susceptible of interception by third parties). Such features provide a security improvement, i.e., where the removal of PII (e.g., facial features) provides an improvement over prior systems because cropped or redacted images, especially ones that may be transmitted over a network (e.g., the Internet), are more secure without including PII information of a user. Accordingly, the systems and methods described herein operate without the need for such non-essential information, which provides an improvement, e.g., a security improvement, over prior system. In addition, the use of cropped images, at least in some embodiments, allows the underlying system to store and/or process smaller data size images, which results in a performance increase to the underlying system as a whole because the smaller data size images require less storage memory and/or processing resources to store, process, and/or otherwise manipulate by the underlying computer system.

In addition, the present disclosure includes applying certain of the claim elements with, or by use of, a particular machine, e.g., an imaging device, which captures images used to train the skin-based learning model and used to determine an image classification corresponding to one or more features of a given user's skin region.

In addition, the present disclosure includes specific features other than what is well-understood, routine, conventional activity in the field, or adding unconventional steps that confine the claim to a particular useful application, e.g., digital imaging and artificial intelligence-based systems and methods for analyzing pixel data of an image of user skin to generate one or more user-specific skin spot classifications.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the system and methods disclosed therein. It should be understood that each Figure depicts an embodiment of a particular aspect of the disclosed system and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and instrumentalities shown, wherein:

FIG. 1 illustrates an example digital imaging and artificial intelligence-based system configured to analyze pixel data of an image of user skin to generate one or more user-specific skin spot classifications, in accordance with various embodiments disclosed herein.

FIG. 2 illustrates an example image and its related pixel data that may be used for training and/or implementing a skin-based learning model, in accordance with various embodiments disclosed herein.

FIG. 3 illustrates an example digital imaging and artificial intelligence-based method for analyzing pixel data of an image of user skin to generate one or more user-specific skin spot classifications, in accordance with various embodiments disclosed herein.

FIGS. 4A-4E illustrates an example image to which a digital imaging and artificial intelligence-based algorithm is applied for analyzing pixel data of an image of user skin to generate one or more user-specific skin spot classifications, in accordance with various embodiments disclosed herein.

FIG. 5 illustrates an example user interface as rendered on a display screen of a user computing device in accordance with various embodiments disclosed herein.

The Figures depict preferred embodiments for purposes of illustration only. Alternative embodiments of the systems and methods illustrated herein may be employed without departing from the principles of the invention described herein.

### DETAILED DESCRIPTION OF THE INVENTION

The systems and methods described herein can be implemented for identifying, classifying, and/or treating skin spots or marks which can be defined as the contrast caused by biological chromophores such as hemoglobin or melanin. These spots may include but not limited to freckles, solar lentigo, melasma, seborrheic keratosis, actinic keratoses, post-inflammatory hyperpigmentation, post-inflammatory erythema, mole, blotchiness, darkened pores. The term “marks” and “spots” can be used interchangeably.

Skin spots or marks can be persistent and there are few over-the-counter remedies that are effective in returning the skin to its original uniformity. Spots or marks are hypothesized to follow a dynamic path as follows. When the skin is subjected to an insult (e.g., acne including comedones, wounds, insect bites), local inflammation starts, and one or more red spots are often formed. The redness and inflammation are generally attributed to the chromophore hemoglobin. With time, the amount of hemoglobin in red spots can change, which alters the spots' appearance. In some cases, increased melanin production is also observed in these red spots. This increase in melanin tends to cause a darkening of the spots' appearance. These spots can naturally heal with time and therefore, hemoglobin and/or melanin in these spots is expected to reduce over time. However, sometimes, especially in more severe cases, the spots may stay on the skin for a longer period. It was found that a composition with hydroxycinnamic acids (HCAs) and niacinamide at a low pH can decrease the melanin and hemoglobin in persistent spots or marks. The disclosure herein describes systems and methods for identification and/or classification of such spots, which allows effective treatment, such as product and/or composition selection and use.

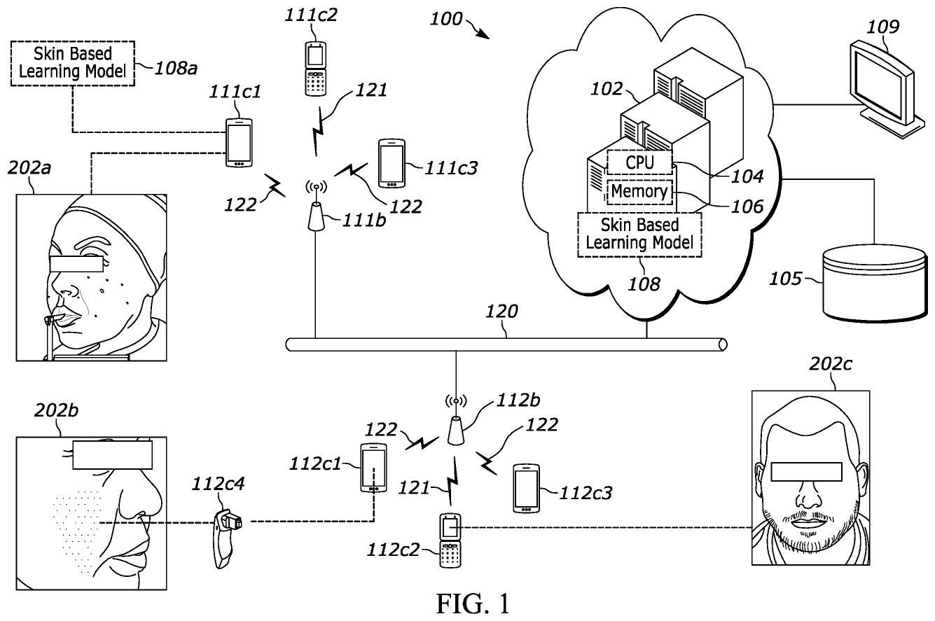

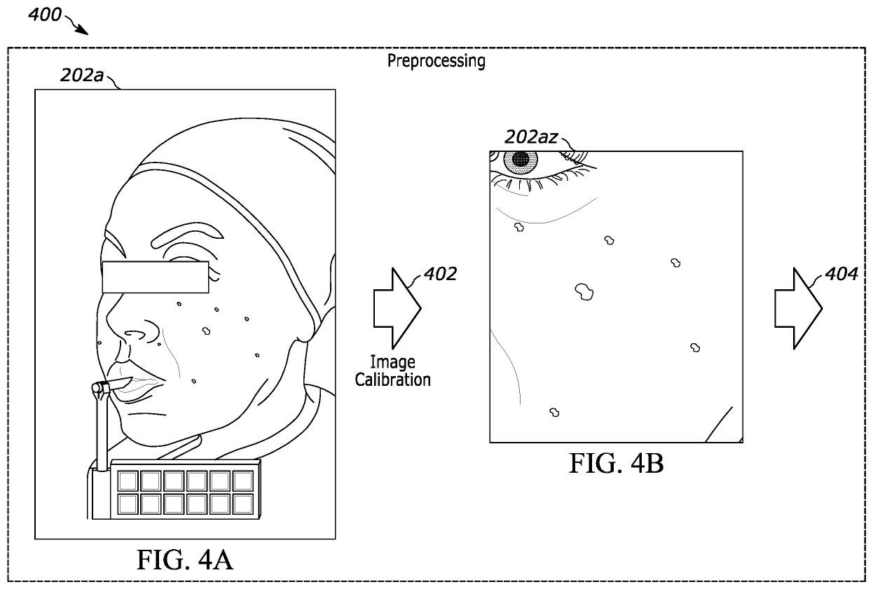

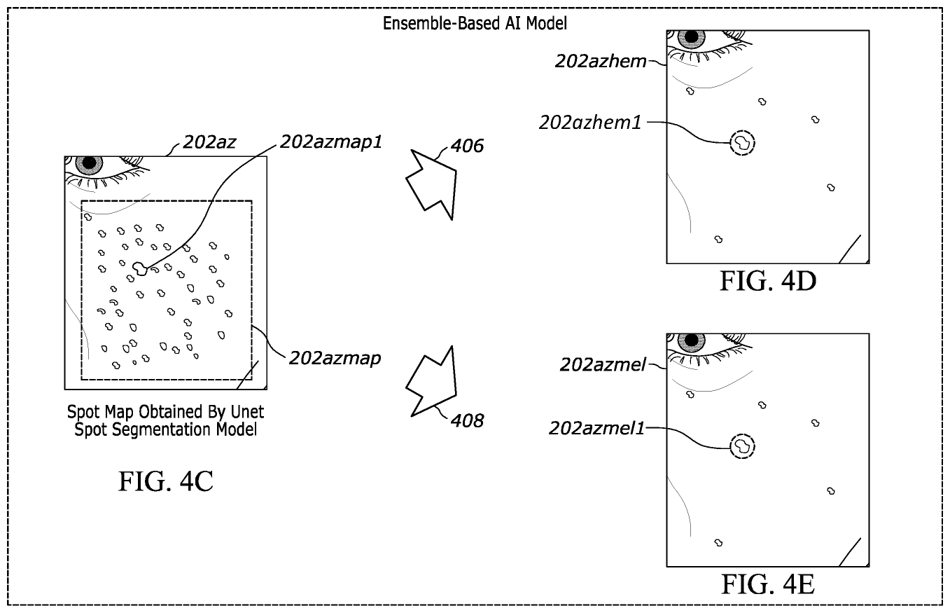

FIG. 1 illustrates an example digital imaging and artificial intelligence-based system configured to analyze pixel data of an image e.g., any one or more of images 202a, 202b, and/or 202c) of user skin to generate one or more user-specific skin spot classifications, in accordance with various embodiments disclosed herein. Generally, as referred to herein, one or more spot classifications may comprise one or more of a hemoglobin type classification (e.g., as depicted herein for the example image of FIG. 4D) or a melanin type classification (e.g., as depicted herein for the example image of FIG. 4E). It is to be understood, however, that additional and/or different types of skin spots may also be analyzed, identified, and/or classified in accordance with the systems and methods herein.

In the example embodiment of FIG. 1, digital imaging and artificial intelligence-based system 100 includes server(s) 102, which may comprise one or more computer servers. In various embodiments server(s) 102 comprise multiple servers, which may comprise multiple, redundant, or replicated servers as part of a server farm. In still further embodiments, server(s) 102 may be implemented as cloud-based servers, such as a cloud-based computing platform. For example, imaging server(s) 102 may be any one or more cloud-based platform(s) such as MICROSOFT AZURE, AMAZON AWS, or the like. Server(s) 102 may include one or more processor(s) 104 (i.e., CPU(s)) as well as one or more computer memories 106. In various embodiments, server(s) 102 may be referred to herein as “imaging server(s).”

Memories 106 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. Memorie(s) 106 may store an operating system (OS) (e.g., Microsoft Windows, Linux, UNIX, etc.) capable of facilitating the functionalities, apps, methods, or other software as discussed herein. Memorie(s) 106 may also store a skin-based learning model 108, which may comprise an artificial intelligence based model, such as a machine learning model, trained on various images (e.g., images 202a, 202b, and/or 202c), as described herein. Additionally, or alternatively, the skin-based learning model 108 may also be stored in database 105, which is accessible or otherwise communicatively coupled to imaging server(s) 102. In addition, memories 106 may also store machine readable instructions, including any of one or more application(s) (e.g., an imaging application as described herein), one or more software component(s), and/or one or more application programming interfaces (APIs), which may be implemented to facilitate or perform the features, functions, or other disclosure described herein, such as any methods, processes, elements or limitations, as illustrated, depicted, or described for the various flowcharts, illustrations, diagrams, figures, and/or other disclosure herein. For example, at least some of the applications, software components, or APIs may be, include, otherwise be part of, an imaging-based machine learning model or component, such as the skin-based learning model 108, where each may be configured to facilitate their various functionalities discussed herein. It should be appreciated that one or more other applications may be envisioned and that are executed by the processor(s) 104.

The processor(s) 104 may be connected to the memories 106 via a computer bus responsible for transmitting electronic data, data packets, or otherwise electronic signals to and from the processor(s) 104 and memories 106 in order to implement or perform the machine-readable instructions, methods, processes, elements or limitations, as illustrated, depicted, or described for the various flowcharts, illustrations, diagrams, figures, and/or other disclosure herein.

Processor(s) 104 may interface with memory 106 via the computer bus to execute an operating system (OS). Processor(s) 104 may also interface with the memory 106 via the computer bus to create, read, update, delete, or otherwise access or interact with the data stored in memories 106 and/or the database 104 (e.g., a relational database, such as Oracle, DB2, MySQL, or a NoSQL based database, such as MongoDB). The data stored in memories 106 and/or database 105 may include all or part of any of the data or information described herein, including, for example, training images and/or user images (e.g., including any one or more of images 202a, 202b, and/or 202c; zoomed, cropped, and/or segmentation related images (e.g., 202azs, 202azs1, 202azs2, etc.); and/or other images and/or information of the user, including demographic, age, race, skin type, or the like, or as otherwise described herein.

Imaging server(s) 102 may further include a communication component configured to communicate (e.g., send and receive) data via one or more external/network port(s) to one or more networks or local terminals, such as computer network 120 and/or terminal 109 (for rendering or visualizing) described herein. In some embodiments, imaging server(s) 102 may include a client-server platform technology such as ASP.NET, Java J2EE, Ruby on Rails, Node.js, a web service or online API, responsive for receiving and responding to electronic requests. The imaging server(s) 102 may implement the client-server platform technology that may interact, via the computer bus, with the memories(s) 106 (including the applications(s), component(s), API(s), data, etc. stored therein) and/or database 105 to implement or perform the machine readable instructions, methods, processes, elements or limitations, as illustrated, depicted, or described for the various flowcharts, illustrations, diagrams, figures, and/or other disclosure herein.

In various embodiments, the imaging server(s) 102 may include, or interact with, one or more transceivers (e.g., WWAN, WLAN, and/or WPAN transceivers) functioning in accordance with IEEE standards, 3GPP standards, or other standards, and that may be used in receipt and transmission of data via external/network ports connected to computer network 120. In some embodiments, computer network 120 may comprise a private network or local area network (LAN). Additionally, or alternatively, computer network 120 may comprise a public network such as the Internet.

Imaging server(s) 102 may further include or implement an operator interface configured to present information to an administrator or operator and/or receive inputs from the administrator or operator. As shown in FIG. 1, an operator interface may provide a display screen (e.g., via terminal 109). Imaging server(s) 102 may also provide I/O components (e.g., ports, capacitive or resistive touch sensitive input panels, keys, buttons, lights, LEDs), which may be directly accessible via, or attached to, imaging server(s) 102 or may be indirectly accessible via or attached to terminal 109. According to some embodiments, an administrator or operator may access the server 102 via terminal 109 to review information, make changes, input training data or images, initiate training of skin-based learning model 108, and/or perform other functions.

As described herein, in some embodiments, imaging server(s) 102 may perform the functionalities as discussed herein as part of a “cloud” network or may otherwise communicate with other hardware or software components within the cloud to send, retrieve, or otherwise analyze data or information described herein.

In general, a computer program or computer based product, application, or code (e.g., the model(s), such as AI models, or other computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) 104 (e.g., working in connection with the respective operating system in memories 106) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective-C, Java, Scala, ActionScript, JavaScript, HTML, CSS, XML, etc.).

As shown in FIG. 1, imaging server(s) 102 are communicatively connected, via computer network 120 to the one or more user computing devices 111c1-111c3 and/or 112c1-112c4 via base stations 111b and 112b. In some embodiments, base stations 111b and 112b may comprise cellular base stations, such as cell towers, communicating to the one or more user computing devices 111c1-111c3 and 112c1-112c4 via wireless communications 121 based on any one or more of various mobile phone standards, including NMT, GSM, CDMA, UMMTS, LTE, 5G, or the like. Additionally, or alternatively, base stations 111b and 112b may comprise routers, wireless switches, or other such wireless connection points communicating to the one or more user computing devices 11c1-111c3 and 112c1-112c4 via wireless communications 122 based on any one or more of various wireless standards, including by non-limiting example, IEEE 802.11a/b/c/g (WIFI), the BLUETOOTH standard, or the like.

Any of the one or more user computing devices 111c1-111c3 and/or 112c1-112c4 may comprise mobile devices and/or client devices for accessing and/or communications with imaging server(s) 102. Such mobile devices may comprise one or more mobile processor(s) and/or an imaging device for capturing images, such as images as described herein (e.g., any one or more of images 202a, 202b, and/or 202c). In various embodiments, user computing devices 111c1-111c3 and/or 112c1-112c3 may comprise a mobile phone (e.g., a cellular phone), a tablet device, a personal data assistance (PDA), or the like, including, by non-limiting example, an APPLE iPhone or iPad device or a GOOGLE ANDROID based mobile phone or table.

In additional embodiments, the user computing device 112c4 may be a portable microscope device such as a dermascope that a user may use to capture detailed images of the user's skin. Specifically, the portable microscope device 112c4 may include a microscopic camera that is configured to capture images (e.g., any one or more of images 202a, 202b, and/or 202c) at an approximately microscopic level of a skin region of a user's skin. For example, unlike any of the user computing devices 11c1-1111c3 and 112c1-112c3, the portable microscope device 112c4 may capture detailed, high-magnification (e.g., 2 megapixels for 60-200 times magnification) images of the user's skin while maintaining physical contact with the user's skin. As a particular example, the portable microscope device 112c4 may be the API 100 SKIN ANALYSIS device, developed by NERA SOLUTIONS LTD. In certain embodiments, the portable microscope device 112c4 may also include a display or user interface configured to display the captured images and/or the results of the image analysis to the user.

Additionally, or alternatively, the portable microscope device 112c4 may be communicatively coupled to a user computing device 112c1 (e.g., a user's mobile phone) via a WIFI connection, a BLUETOOTH connection, and/or any other suitable wireless connection, and the portable microscope device 112c4 may be compatible with a variety of operating platforms (e.g., Windows, iOS, Android, etc.). Thus, the portable microscope device 112c4 may transmit the captured images to the user computing device 112c1 for analysis and/or display to the user. Moreover, the portable microscope device 112c4 may be configured to capture high-quality video of a user's skin, and may stream the high-quality video of the user's skin to a display of the portable microscope device 112c4 and/or a communicatively coupled user computing device 112c1 (e.g., a user's mobile phone). In certain additional embodiments, the components of each of the portable microscope device 112c4 and the communicatively connected user computing device 112c1 may be incorporated into a singular device.

In additional embodiments, user computing devices 11c1-111c3 and/or 112c1-112c3 may comprise a retail computing device. A retail computing device may comprise a user computer device configured in a same or similar manner as a mobile device, e.g., as described herein for user computing devices 111c1-111c3, including having a processor and memory, for implementing, or communicating with (e.g., via server(s) 102), a skin-based learning model 108 as described herein. Additionally, or alternatively, a retail computing device may be located, installed, or otherwise positioned within a retail environment to allow users and/or customers of the retail environment to utilize the digital imaging and artificial intelligence-based systems and methods on site within the retail environment. For example, the retail computing device may be installed within a kiosk for access by a user. The user may then upload or transfer images (e.g., from a user mobile device) to the kiosk to implement the digital imaging and artificial intelligence-based systems and methods described herein. Additionally, or alternatively, the kiosk may be configured with a camera to allow the user to take new images (e.g., in a private manner where warranted) of himself or herself for upload and transfer. In such embodiments, the user or consumer himself or herself would be able to use the retail computing device to receive and/or have rendered a user-specific electronic spot classification, as described herein, on a display screen of the retail computing device.

Additionally, or alternatively, the retail computing device may be a mobile device (as described herein) as carried by an employee or other personnel of the retail environment for interacting with users or consumers on site. In such embodiments, a user or consumer may be able to interact with an employee or otherwise personnel of the retail environment, via the retail computing device (e.g., by transferring images from a mobile device of the user to the retail computing device or by capturing new images by a camera of the retail computing device), to receive and/or have rendered a user-specific electronic skin classification, as described herein, on a display screen of the retail computing device.

In various embodiments, the one or more user computing devices 111c1-111c3 and/or 112c1-112c4 may implement or execute an operating system (OS) or mobile platform such as Apple's iOS and/or Google's Android operation system. Any of the one or more user computing devices 111c1-111c3 and/or 112c1-112c4 may comprise one or more processors and/or one or more memories for storing, implementing, or executing computing instructions or code, e.g., a mobile application or a home or personal assistant application, as described in various embodiments herein. As shown in FIG. 1, skin-based learning model 108a and/or an imaging application as described herein, or at least portions thereof, may also be stored locally on a memory of a user computing device (e.g., user computing device 111c1). In some aspects, skin-based learning model 108a as installed on a computing device may comprise the same skin-based learning model 108 as installed on server(s) 102. Additionally, or alternatively, skin-based learning model 108a may comprise a portion of skin-based learning model 108 as installed on server(s) 102. It is to be understood that in some aspects, skin-based learning model may be installed wholly at user computing device, wholly at server(s) 102, or partially on user computing device and partially on server(s) 102 where communication between skin-based learning model 108a and skin-based learning model 108 occurs through computer network 120. Generally, when skin-based learning model is referred to herein, it refers to one or both of skin-based learning model 108 and/or -based learning model 108a.

User computing devices 111c1-111c3 and/or 112c1-112c4 may comprise a wireless transceiver to receive and transmit wireless communications 121 and/or 122 to and from base stations 111b and/or 112b. In various embodiments, pixel based images (e.g., images 202a, 202b, and/or 202c) may be transmitted via computer network 120 to imaging server(s) 102 for training of model(s) (e.g., skin-based learning model 108) and/or imaging analysis as described herein.

In addition, the one or more user computing devices 111c1-111c3 and/or 112c1-112c4 may include an imaging device and/or digital video camera for capturing or taking digital images and/or frames (e.g., which can be any one or more of images 202a, 202b, and/or 202c). Each digital image may comprise pixel data for training or implementing model(s), such as AI or machine learning models, as described herein. For example, an imaging device and/or digital video camera of, e.g., any of user computing devices 111c1-111c3 and/or 112c1-112c4, may be configured to take, capture, or otherwise generate digital images (e.g., pixel based images 202a, 202b, and/or 202c) and, at least in some embodiments, may store such images in a memory of a respective user computing devices. Additionally, or alternatively, such digital images may also be transmitted to and/or stored on memorie(s) 106 and/or database 105 of server(s) 102.

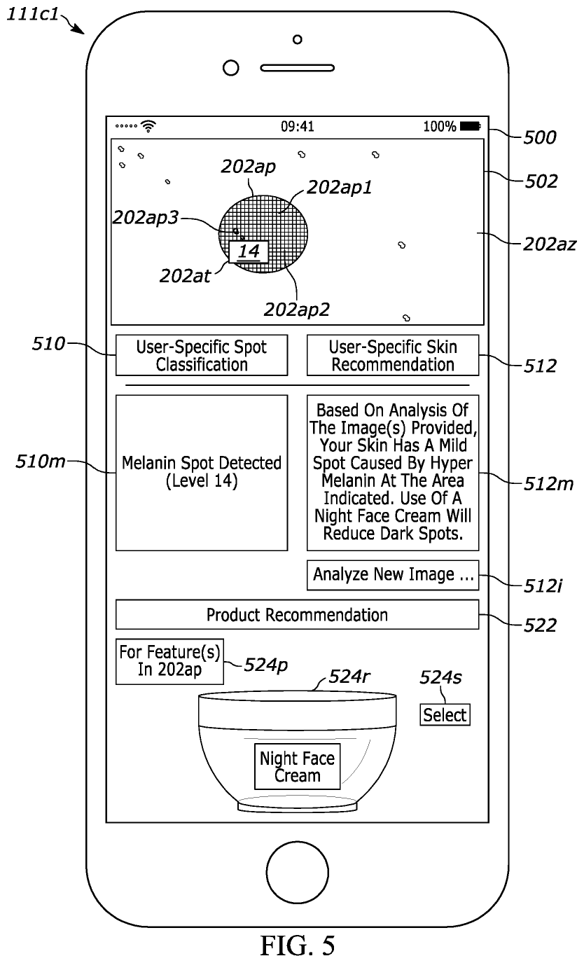

Still further, each of the one or more user computer devices 111c1-111c3 and/or 112c1-112c4 may include a display screen for displaying graphics, images, text, spot classifications, skin products, data, pixels, features, and/or other such visualizations or information as described herein. In various embodiments, graphics, images, text, spot classifications, skin products, data, pixels, features, and/or other such visualizations or information may be received from imaging server(s) 102 for display on the display screen of any one or more of user computer devices 111c1-111c3 and/or 112c1-112c4. Additionally, or alternatively, a user computer device, e.g., as described herein for FIG. 5, may comprise, implement, have access to, render, or otherwise expose, at least in part, an interface or a guided user interface (GUI) for displaying text and/or images on its display screen.

In some embodiments, computing instructions and/or applications executing at the server (e.g., server(s) 102) and/or at a mobile device (e.g., mobile device 111c1) may be communicatively connected for analyzing pixel data of an image of user skin to generate one or more user-specific skin spot classifications, as described herein. For example, one or more processors (e.g., processor(s) 104) of server(s) 102 may be communicatively coupled to a mobile device via a computer network (e.g., computer network 120). In such embodiments, an imaging app may comprise a server app portion configured to execute on the one or more processors of the server (e.g., server(s) 102) and a mobile app portion configured to execute on one or more processors of the mobile device (e.g., any of one or more user computing devices 11c1-111c3 and/or 112c1-112c3) and/or standalone imaging device (e.g., user computing device 112c4). In such embodiments, the server app portion is configured to communicate with the mobile app portion. The server app portion or the mobile app portion may each be configured to implement, or partially implement, one or more of: (1) receiving the image captured by the imaging device; (2); determining the at least one spot classification of the user's skin region; (3) generating the user-specific spot classification; and/or (4) transmitting a user-specific recommendation the computing device app portion.

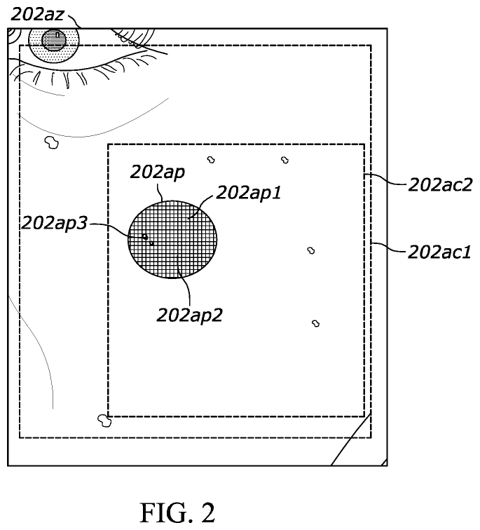

FIG. 2 illustrates an example image 202az and its related pixel data that may be used for training and/or implementing a skin-based learning model, in accordance with various embodiments disclosed herein. In various embodiments, as shown for FIG. 2, image 202az may be an image captured by a user. In the embodiment, image 202az represents and is depicted as a zoomed or cropped version of image 202a of FIG. 1. Image 202az (as well as images 202a, 202b and/or 202c) may be transmitted to server(s) 102 via computer network 120, as shown for FIG. 1. It is to be understood that such images may be captured by the users themselves or, additionally or alternatively, others, such as a retailer, etc., where such images are used and/or transmitted on behalf of a user.

More generally, digital images, such as example images 202a, 202b, and 202c, may be collected or aggregated at imaging server(s) 102 and may be analyzed by, and/or used to train, a skin-based learning model (e.g., an AI model such as a machine learning imaging model as described herein). Each of these images may comprise pixel data comprising feature data and corresponding to skin regions of respective users, within the respective image. The pixel data may be captured by an imaging device of one of the user computing devices (e.g., one or more user computer devices 111c1-111c3 and/or 112c1-112c4).

With respect to digital images as described herein, pixel data (e.g., pixel data 202ap of FIG. 2) comprises individual points or squares of data within an image, where each point or square represents a single pixel (e.g., each of pixel 202ap1, pixel 202ap2, and pixel 202ap3) within an image. Each pixel may be at a specific location within an image. In addition, each pixel may have a specific color (or lack thereof). Pixel color, may be determined by a color format and related channel data associated with a given pixel. For example, a popular color format is a 1976 CIELAB (also referenced herein as the “CIE L*-a*-b*” or simply “L*a*b*” color format) color format that is configured to mimic the human perception of color. Namely, the L*a*b* color format is designed such that the amount of numerical change in the three values representing the L*a*b* color format (e.g., L*, a*, and b*) corresponds roughly to the same amount of visually perceived change by a human. This color format is advantageous, for example, because the L*a*b* gamut (e.g., the complete subset of colors included as part of the color format) includes both the gamuts of Red (R), Green (G), and Blue (B) (collectively RGB) and Cyan (C), Magenta (M), Yellow (Y), and Black (K) (collectively CMYK) color formats.

In the L*a*b* color format, color is viewed as point in three dimensional space, as defined by the three-dimensional coordinate system (L*, a*, b*), where each of the L* data, the a* data, and the b* data may correspond to individual color channels, and may therefore be referenced as channel data. In this three-dimensional coordinate system, the L* axis describes the brightness (luminance) of the color with values from 0 (black) to 100 (white). The a* axis describes the green or red ratio of a color with positive a* values (+a*) indicating red hue and negative a* values (−a*) indicating green hue. The b* axis describes the blue or yellow ratio of a color with positive b* values (+b*) indicating yellow hue and negative b* values (−b*) indicating blue hue. Generally, the values corresponding to the a* and b* axes may be unbounded, such that the a* and b* axes may include any suitable numerical values to express the axis boundaries. However, the a* and b* axes may typically include lower and upper boundaries that range from approximately 150 to −150. Thus, in this manner, each pixel color value may be represented as a three-tuple of the L*, a*, and b* values to create a final color for a given pixel.

As another example, a popular color format includes the red-green-blue (RGB) format having red, green, and blue channels. That is, in the RGB format, data of a pixel is represented by three numerical RGB components (Red, Green, Blue), that may be referred to as a channel data, to manipulate the color of pixel's area within the image. In some implementations, the three RGB components may be represented as three 8-bit numbers for each pixel. Three 8-bit bytes (one byte for each of RGB) may be used to generate 24-bit color. Each 8-bit RGB component can have 256 possible values, ranging from 0 to 255 (i.e., in the base 2 binary system, an 8-bit byte can contain one of 256 numeric values ranging from 0 to 255). This channel data (R, G, and B) can be assigned a value from 0 to 255 that can be used to set the pixel's color. For example, three values like (250, 165, 0), meaning (Red=250, Green=165, Blue=0), can denote one Orange pixel. As a further example, (Red=255, Green=255, Blue=0) means Red and Green, each fully saturated (255 is as bright as 8 bits can be), with no Blue (zero), with the resulting color being Yellow. As a still further example, the color black has an RGB value of (Red=0, Green=0, Blue=0) and white has an RGB value of (Red=255, Green=255, Blue=255). Gray has the property of having equal or similar RGB values, for example, (Red=220, Green=220, Blue=220) is a light gray (near white), and (Red=40, Green=40, Blue=40) is a dark gray (near black).

In this way, the composite of three RGB values creates a final color for a given pixel. With a 24-bit RGB color image, using 3 bytes to define a color, there can be 256 shades of red, and 256 shades of green, and 256 shades of blue. This provides 256×256×256, i.e., 16.7 million possible combinations or colors for 24 bit RGB color images. As such, a pixel's RGB data value indicates a degree of color or light each of a Red, a Green, and a Blue pixel is comprised of. The three colors, and their intensity levels, are combined at that image pixel, i.e., at that pixel location on a display screen, to illuminate a display screen at that location with that color. In is to be understood, however, that other bit sizes, having fewer or more bits, e.g., 10-bits, may be used to result in fewer or more overall colors and ranges.

As a whole, the various pixels, positioned together in a grid pattern (e.g., pixel data 202ap), form a digital image or portion thereof. A single digital image can comprise thousands or millions of pixels. Images can be captured, generated, stored, and/or transmitted in a number of formats, such as JPEG, TIFF, PNG and GIF. These formats use pixels to store or represent the image.

With reference to FIG. 2, example image 202az illustrates a skin region of a user or individual. More specifically, image 202az comprises pixel data, including pixel data 202ap defining the skin region of the user's or individual's skin. Pixel data 202ap includes a plurality of pixels including pixel 202ap1, pixel 202ap2, and pixel 202ap3. In example image 202a, each of pixel 202ap1, pixel 202ap2, and pixel 202ap3 are representative of features of skin corresponding to image classifications of a skin region. Generally, in various embodiments, features of the skin or otherwise skin region of a user may comprise one or more of spots related to hemoglobin and/or spots related to melanin. Each of these features may be determined from or otherwise based on one or more pixels in a digital image (e.g., image 202az). For example, with respect to image 202az, each of pixels 202ap1 and 202ap2 may be relatively light pixels (e.g., pixels with relatively high L* values) and/or relatively yellow pixels (e.g., pixels with relatively high or positive b* values) positioned within pixel data 202ap in a region of the user's skin, which may be indicative of regular or more common values of the user's skin. Pixel 202ap3 however, may comprise darker pixels (e.g., with negative or lower relative *L values) and/or redder pixels (e.g., positive or higher relative a* values), which may be indicative of a melanin or hemoglobin related spot, respectively, at that location in the image of the user's skin. Such pixel features may be used to train skin-based learning model (e.g., skin-based learning model 108) to generate a user-specific skin recommendation designed to address at least one spot feature identifiable within the pixel data comprising the portion of the skin region of the user. In addition to pixels 202ap1, 202ap2, and 202ap3, pixel data 202ap includes various other pixels including remaining portions of the user's skin, including various other skin regions and/or portions of skin that may be analyzed and/or used for training of model(s), and/or analysis by used of already trained models, such as skin-based learning model 108 as described herein. For example, pixel data 202ap further includes pixels representative of features of spots, and, in various aspects, in addition to the color of a spot, the grouping of such pixels at a particular location in the image, where such pixels having similar L*a*b* and/or RGB values, provides training information for spot classification as described herein.

A digital image, such as a training image, an image as submitted by users, or otherwise a digital image (e.g., any of images 202a, 202b, and/or 202c), may be or may comprise a cropped image. Generally, a cropped image is an image with one or more pixels removed, deleted, or hidden from an originally captured image. In some aspects, each image of the one or more of the plurality of training images e.g., any of images 202a, 202b, and/or 202c) or the image of the user comprises at least one cropped image depicting the skin region having a single instance of a spot feature. For example, with reference to FIG. 2, image 202az represents at least a portion of an original image. Cropped portion 202ac1 represents a first cropped portion of image 202az that removes portions of the user's skin (outside of cropped portion 202ac1) that may not include readily identifiable spot features. As a further example, cropped portion 202ac2 represents a second cropped portion of image 202az that removes portions of the image (outside of cropped portion 202ac2) that may not include spot features that are as readily identifiable as the features included in the cropped portion 202ac2, and may therefore be less useful as training data. In various embodiments, analyzing and/or use of cropped images for training yields improved accuracy of a skin-based learning model. It also improves the efficiency and performance of the underlying computer system in that such system processes, stores, and/or transfers smaller size digital images. Still further, images may be sent as cropped or that otherwise include extracted or depicted skin regions of a user without depicting personal identifiable information (PII) of the user. Such cropped images provide a security improvement, i.e., where the removal of PII provides an improvement over prior systems because cropped or redacted images, especially ones that may be transmitted over a network (e.g., the Internet), are more secure without including PII information of a user. Importantly, the systems and methods described herein may operate without the need for such non-essential information, which provides an improvement, e.g., a security and a performance improvement, over conventional systems. Moreover, while FIG. 2 may depict and describe a cropped image, it is to be understood, however, that other image types including, but not limited to, original, non-cropped images (e.g., original image 202a) and/or other types/sizes of cropped images (e.g., cropped portion 202ac1 of image 202az) may be used or substituted as well.

It is to be understood that the disclosure for image 202az of FIG. 2 applies the same or similarly for other digital images described herein, including, for example, images 202a, 202b, and/or 202c, where such images also comprise pixels that may be analyzed and/or used for training of model(s) as described herein.

In addition, digital images of a user's skin, as described herein, may depict various skin features, which may be used to train a skin-based learning model across a variety of different users having a variety of different skin features. For example, as illustrated for images 202a, 202b, and 202c, the skin regions of these users comprise skin features (e.g., spots) of the user's skin regions identifiable with the pixel data of the respective images. These skin features include, for example, features indicative of hemoglobin and/or melanin, which can comprise discrete skin regions or features (e.g., spots) at one or more locations distributed across the user's skin.

In various embodiments, digital images (e.g., images 202a, 202b, and 202c), whether used as training images depicting individuals, or used as images depicting users or individuals for analysis and/or spot classification, may comprise multiple angles or perspectives depicting skin of each of the respective individual or the user. That is, each image of the one or more of the plurality of training images or the image of a user may comprise multiple angles or perspectives depicting skin regions of the respective individuals or the user. The multiple angles or perspectives may include different views, positions, closeness of the user and/or backgrounds, lighting conditions, or otherwise environments in which the user is positioned against in a given image. For example, FIG. 1 includes skin images (e.g., 202a, 202b, and 202c) that depict skin regions of respective individuals and/or users and are captured using different lighting conditions (e.g., visible, UV) at different angles. Such images maybe used for training a skin-based learning model, or for analysis, and/or user-specific spot classifications, as described herein.

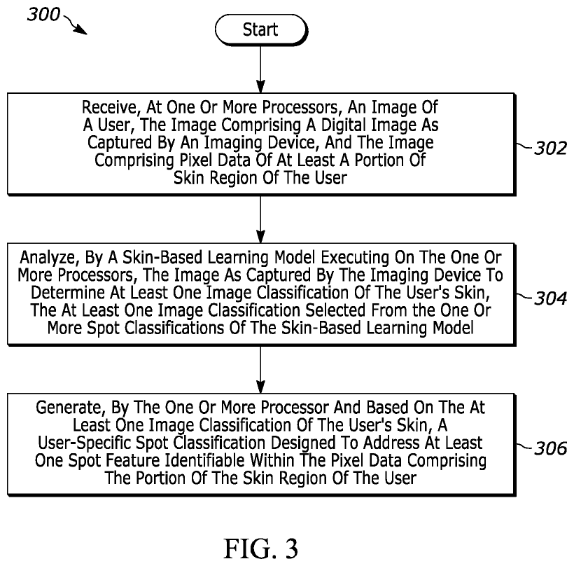

FIG. 3 illustrates an example digital imaging and artificial intelligence-based method 300 300 for analyzing pixel data of an image of user skin to generate one or more user-specific skin spot classifications, in accordance with various embodiments disclosed herein. At block 302, method 300 may comprise receiving, at one or more processors, an image of a user. The image may comprise a digital image (e.g., any of images 202a/202az, 202b, and/or 202c) as captured by an imaging device (e.g., a digital camera of the mobile device 111c1). Further, the image may comprise pixel data of at least a portion of a skin region of the user. In various aspects, the one or more processors may comprise processor(s) 104 of server(s) 102. Additionally, or alternatively, the one or more processors may comprise a processor of a mobile device (e.g., computing device 111c1). Images, as used with the method 300, and more generally as described herein, are pixel-based images as captured by an imaging device (e.g., an imaging device of user computing device 111c1). In some embodiments an image may comprise or refer to a plurality of images such as a plurality of images (e.g., frames) as collected using a digital video camera. Frames comprise consecutive images defining motion, and can comprise a movie, a video, or the like.

At block 304, method 300 further comprises analyzing, by a skin-based learning model (e.g., skin-based learning model 108) executing on the one or more processors, the image as captured by the imaging device to determine at least one spot classification of the user's skin. In the example of FIG. 3, the at least one spot classification is selected from the one or more spot classifications of the skin-based learning model (e.g., skin-based learning model 108). In various aspects, the skin-based learning model (e.g., skin-based learning model 108) is trained with pixel data of a plurality of training images (e.g., any of images 202a/202az, 202b, and/or 202c) depicting skin of respective individuals. The skin-based learning model, once trained, is configured to output one or more spot classifications corresponding to one or more spot features (e.g., hemoglobin or melanin) of skin regions of the respective individuals.

In various aspects the skin-based learning model (e.g., skin-based learning model 108) is an artificial intelligence (AI) based model trained with at least one AI algorithm. Training of the skin-based learning model 108 involves image analysis of the training images to configure weights of the skin-based learning model 108, and its underlying algorithm (e.g., machine learning or artificial intelligence algorithm) used to predict and/or classify future images. For example, in various embodiments herein, generation of the skin-based learning model 108 involves training the skin-based learning model 108 with the plurality of training images (e.g., images 202a, 202b, 202c) of a plurality of individuals, where each of the training images comprise pixel data and depict skin regions or otherwise skin of respective individuals. In some embodiments, one or more processors of a server or a cloud-based computing platform (e.g., imaging server(s) 102) may receive the plurality of training images of the plurality of individuals via a computer network (e.g., computer network 120). In such embodiments, the server and/or the cloud-based computing platform may train the skin-based learning model with the pixel data of the plurality of training images. Additionally, in some aspects, skin-based learning model may be further trained with user demographic data (e.g., data indicating race, skin color, etc.) and environment data (e.g., amount of sunshine, geography, weather conditions, etc.) of the respective users. In such aspects, spot classification(s), as generated by the skin-based learning model, may be further based on user demographic data and environment data as provided by a given user.

In various embodiments, a machine learning imaging model, as described herein (e.g. skin-based learning model 108), may be trained using a supervised or unsupervised machine learning program or algorithm. The machine learning program or algorithm may employ a neural network, which may be a convolutional neural network, a vision transformer, a deep learning neural network, or a combined learning module or program that learns in two or more features or feature datasets (e.g., pixel data) in a particular areas of interest. The machine learning programs or algorithms may also include natural language processing, semantic analysis, automatic reasoning, regression analysis, support vector machine (SVM) analysis, decision tree analysis, random forest analysis, K-Nearest neighbor analysis, naïve Bayes analysis, clustering, reinforcement learning, and/or other machine learning algorithms and/or techniques. In some embodiments, the artificial intelligence and/or machine learning based algorithms may be included as a library or package executed on imaging server(s) 102. For example, libraries may include the TENSORFLOW based library, the PYTORCH library, and/or the SCIKIT-LEARN Python library.

Machine learning may involve identifying and recognizing patterns in existing data (such as identifying features of skin, such as spot and/or color or discoloration related features, in the pixel data of image as described herein) in order to facilitate making predictions or identification for subsequent data (such as using the model on new pixel data of a new image in order to determine or generate a user-specific spot classification designed to address at least one feature identifiable within the pixel data comprising the at least the portion of a skin region of a user).

Machine learning model(s), such as the skin-based learning model described herein for some embodiments, may be created and trained based upon example data (e.g., “training data” and related pixel data) inputs or data (which may be termed “features” and “labels”) in order to make valid and reliable predictions for new inputs, such as testing level or production level data or inputs. In supervised machine learning, a machine learning program operating on a server, computing device, or otherwise processor(s), may be provided with example inputs (e.g., “features”) and their associated, or observed, outputs (e.g., “labels”) in order for the machine learning program or algorithm to determine or discover rules, relationships, patterns, or otherwise machine learning “models” that map such inputs (e.g., “features”) to the outputs (e.g., labels), for example, by determining and/or assigning weights or other metrics to the model across its various feature categories. Such rules, relationships, or otherwise models may then be provided subsequent inputs in order for the model, executing on the server, computing device, or otherwise processor(s), to predict, based on the discovered rules, relationships, or model, an expected output.

In unsupervised machine learning, the server, computing device, or otherwise processor(s), may be required to find its own structure in unlabeled example inputs, where, for example multiple training iterations are executed by the server, computing device, or otherwise processor(s) to train multiple generations of models until a satisfactory model, e.g., a model that provides sufficient prediction accuracy when given test level or production level data or inputs, is generated.

Supervised learning and/or unsupervised machine learning may also comprise retraining, relearning, or otherwise updating models with new, or different, information, which may include information received, ingested, generated, or otherwise used over time. The disclosures herein may use one or both of such supervised or unsupervised machine learning techniques.

In various embodiments, a skin-based learning model (e.g., skin-based learning model 108) may be trained, by one or more processors (e.g., one or more processor(s) 104 of server(s) 102 and/or processors of a computer user device, such as a mobile device) with the pixel data of a plurality of training images (e.g., any of images 202a, 202b, and/or 202c). In various embodiments, a skin-based learning model (e.g., skin-based learning model 108) is configured or trained to output one or more features of a user's skin or skin regions for a given image. In these embodiments, the one or more features of skin or skin regions may differ based on one or more user demographics and/or ethnicities of the respective individuals represented in the respective training images, e.g., as typically associated with, or otherwise naturally occurring for, different races, genomes, and/or geographic locations associated with such demographics and/or ethnicities. Still further, the skin-based learning model (e.g., skin-based learning model 108) may generate a user-specific spot classification of each respective individual represented in the respective training images based on the ethnicity and/or demographic value of the respective individual.

In various embodiments, image analysis may include training a machine learning based model (e.g., the skin-based learning model 108) on pixel data of images depicting skin or skin regions of respective individuals. Additionally, or alternatively, image analysis may include using a machine learning imaging model, as previously trained, to determine, based on the pixel data (e.g., including their L*, a*, and b* values and/or RGB values) one or more images of the individual(s), an image classification of the user's skin or skin region. For example, the weights of the model may be trained via analysis of various L*a*b* values of individual pixels of a given image. For example, dark or low L* values (e.g., a pixel with an L* value less than 50) may indicate regions of an image where hemoglobin and/or melanin is present. Likewise, a slightly lighter L* values (e.g., a pixel with an L* value greater than 50) may indicate the absence of melanin or hemoglobin. Still further, high/low a* values may indicate areas of the skin containing more/less melanin and/or hemoglobin. Together, when a pixel having skin toned L*a*b* values is positioned within a given image, or is otherwise surrounded by, a group or set of pixels having melanin and/or hemoglobin toned colors, then a skin-based learning model (e.g., skin-based learning model 108) can determine an image or otherwise spot classification of a user's skin region and related spots, as identified within the given image. In this way, pixel data (e.g., detailing skin regions of skin of respective individuals) of 10,000s training images may be used to train or use a machine learning imaging model to determine an image classification of the user's skin region, and various spot classifications thereof.

Further with reference to FIG. 3, at block 306, method 300 further comprises generating by the one or more processors and based on the at least one spot classification of the user's skin, a user-specific skin recommendation designed to address at least one spot feature identifiable within the pixel data comprising the portion of the skin region of the user. In various embodiments, computing instructions of the imaging app when executed by one or more processors, may cause the one or more processors to generate a skin quality code as determined based on the user-specific spot classification designed to address the at least one spot feature identifiable within the pixel data comprising the portion of the skin region of the user. In some aspects, the skin quality code may comprise an ID within a range or class of IDs for a particular skin type classification. The IDs or codes may be arranged in a degree of severity for a given type, e.g., lower values indicate less sever melanin and/or hemoglobin on the skin and vice versa. These skin quality codes may include a L*a*b* value, RGB value, a percentage of melanin and/or hemoglobin detected in the skin, and/or any other suitable code. For example, in these embodiments, the user-specific spot classification may include an average/sum value corresponding to the respective IDs/values associated with each feature/attribute analyzed as part of the skin-based learning model (e.g., skin-based learning model 108), such as a ratio of hemoglobin-to-melanin as detected, identified, or classified by skin-based learning model 108. To illustrate, if a user a high skin quality code (e.g., classification of a value of 7 for hemoglobin or 17 for melanin), then the user may receive a user-specific spot classification of “poor” or “unhealthy.” By contrast, if a user receives low scores (e.g., 1 or 11, respectively), then the user may receive a user-specific spot classification of “good” or “healthy.”

In still further embodiments, computing instructions of the imaging app may further cause one or more processors to record the image of the user as captured by the imaging device at a first time for tracking changes to user's skin region over time. The second image may include pixel data of at least a portion of a skin region of the user's skin. The computing instructions may also cause the one or more processors to analyze, by the skin-based learning model, the second image captured by the imaging device to determine, at the second time, a second image classification of the user's skin region as selected from the one or more image classifications of the skin-based learning model, and generate, based on a comparison of the image and the second image and/or the image classification and the second image classification of the user's skin region, a new user-specific spot classification regarding at least one spot feature identifiable or lack thereof within the pixel data of the second image comprising at least the portion the skin region of the user.

In additional aspects, the at least one user-specific skin recommendation is displayed on a display screen of the computing device with instructions for treating the at least one spot feature identifiable in the pixel data comprising the portion of the skin region of the user. In some aspects, the user-specific skin recommendation may be rendered on the display screen in real-time or near-real time, during, or after receiving, the image of the user. For example, the instructions may provide the user with information (e.g., avoid direct sun exposure) in order reduce or eliminate hyper melanin production at the skin region identifiable within the image. This aspect if further exemplified herein by FIG. 5.

In still further aspects, the at least one user-specific spot recommendation comprises a product recommendation for a manufactured product. The manufactured product may comprise a pharmaceutical, therapeutic, or other product for treating the at least one spot feature identifiable in the pixel data. For example, the product may comprise a composition, such as a cream, with hydroxycinnamic acids (HCAs) and niacinamide at a low pH can decrease the melanin and hemoglobin in persistent spots or marks. In some aspects, a product based user-specific skin recommendation may displayed on the display screen of the computing device with instructions for treating, with the manufactured product, the at least one spot feature identifiable in the pixel data comprising the portion of a skin region of the user. Still further, in some aspects, computing instructions may further cause the one or more processors to initiate, based on the at least one user-specific skin recommendation, the manufactured product for shipment to the user.

With regard to manufactured product recommendations, in some embodiments, one or more processors (e.g., imaging server(s) 102 and/or a user computing device, such as user computing device 111c1) may generate a modified image based on the at least one image of the user, e.g., as originally received. In such embodiments, the modified image may depict a rendering of how the user's skin or skin regions are predicted to appear after treating the at least one feature with the manufactured product. For example, the modified image may be modified by updating, smoothing, or changing colors of the pixels of the image to represent a possible or predicted change after treatment of the at least one feature within the pixel data with the manufactured product. The modified image may then be rendered on the display screen of the user computing device (e.g., user computing device 111c1).

In various aspects, a user-specific skin recommendation may be generated by a user computing device (e.g., user computing device 111c1) and/or by a server (e.g., imaging server(s) 102). For example, in some embodiments imaging server(s) 102, as described herein for FIG. 1, may analyze a user image remote from a user computing device to determine an image classification of the user's skin, the user-specific spot classification designed to address at least one feature identifiable within the pixel data comprising the at least the portion of a skin region of the user, and/or the user-specific skin recommendation itself. For example, in such embodiments imaging server or a cloud-based computing platform (e.g., imaging server(s) 102) receives, across computer network 120, the at least one image comprising the pixel data of at the least a portion of a skin region of the user's skin. The server or a cloud-based computing platform may then execute skin-based learning model (e.g., skin-based learning model 108) and generate, based on output of the skin-based learning model (e.g., skin-based learning model 108), the user-specific skin recommendation. The server or a cloud-based computing platform may then transmit, via the computer network (e.g., computer network 120), the user-specific skin recommendation to the user computing device for rendering on the display screen of the user computing device. For example, and in various embodiments, the at least one user-specific skin recommendation may be rendered on the display screen of the user computing device in real-time or near-real time, during, or after receiving, the image having the skin region of the user's skin.

FIGS. 4A-4E illustrates an example image (e.g., image 202a) to which a digital imaging and artificial intelligence-based algorithm 400 is applied for analyzing pixel data of an image of user skin to generate one or more user-specific skin spot classifications, in accordance with various embodiments disclosed herein. In some aspects, the algorithm of FIGS. 4A-4E may be implemented as, or as part of, method 300 as described herein.

As shown for FIG. 4A, an image is input or otherwise selected for processing by the algorithm 400. In the example, the image of FIG. 4A is image 202a. At step 402, image calibration may be applied. More particularly, image calibration may comprise a preprocessing step including applying a chromatic adaptation algorithm and/or a white balancing (e.g., deep learning white balancing) algorithm to the image (e.g., image 202a) in order to prepare the image for input into the skin-based learning model (e.g., skin-based learning model 108). Such preprocessing can eliminate data noise or otherwise extraneous elements to improve the accuracy of the skin-based learning model (e.g., skin-based learning model 108). For example, image calibration comprising chromatic adaptation may comprise taking a negative, or other variant, of the original image in order to remove classification anomalies caused by skin color differences. As a further example, image calibration may comprise white balancing, where the pixel values are enhanced, reduced, or otherwise changed from one image to another in order to remove classification anomalies caused by skin color differences.

The image calibration algorithm can be applied to both input images for classification and to training images used to train a model. For example, each of a plurality of training images (e.g., any one or more of images 202a, 202b, and/or 202c, etc.) have image calibration applied to alter the images enhance spot classification for training purposes. Then, the image calibration algorithm can be applied to later input images of the user prior to analyzing, with the skin-based learning model, the image of the user in order to have a same or similar image type input into the trained model, where all images used for training and later input have the same image calibration algorithm applied thereto.

In some aspects, image calibration may also include cropping or zooming an original image to remove extraneous features, and thus reduce file size. For example, as shown for FIG. 4B, image 202az is a cropped or zoomed variant of image 202a, where image 202az was obtained by application of image calibration in step 402.

At step 404, the preprocessed image (e.g., image 202az) may be input into skin-based learning model 108. In the example of FIGS. 4A-4E, skin-based learning model 108 is an ensemble-based AI model, e.g., such as a transformer model, comprising (i) a segmentation model configured to generate a segmentation mapping of one or more spots in a skin region of an image, and (ii) a prediction or classification model configured to analyze the pixel data of the segmentation mapping of one or more spots. In one example, the segmentation model may comprise a UNET based segmentation model configured to detect or otherwise determine possible regions on a user's skin that may have spots. In particular, the segmentation model may generate a spot map 202azmap that maps one or more possible spots or segments on the user's skin. The segments may comprise groupings of one or more pixels within the image (e.g., image 202az) having the same and/or similar pixel values (e.g., the same and/or similar LAB and/or RGB values). For example, as shown in FIG. 4C, segmentation model has been executed to determine spot map 202azmap, which comprises various segments within the image with possible skin spots. This includes spot segment 202azmap1, which comprises pixel 202ap3 (having discoloration or otherwise a skin spot) as described herein. Thus, at step 404 computing instructions (e.g., of an imaging app), when executed by the one or more processors (e.g., processors 104 or processors of a computing device 111c1), may, at step 402, cause the one or more processors to generate a user-specific segmentation mapping of one or more spots in the portion of the skin region of the user identifiable in the image of the user.

The prediction or classification model of the ensemble-based AI model may comprise any one or more of a machine learning model, regression equation, or a deep learning model. For example, the machine learning model may comprise a principal component model trained on multiple spot maps (as output by the segmentation model) to determine the principal components (e.g., the features, such as pixel feature) that have the highest predictive and/or classification value(s) for accurately identifying skin spots or related spot segments (e.g., 202azmap1) within the spot map (e.g., 202azmap). Similarly, a machine learning model may comprise a regression equation having one or more independent variables with values trained to identify a highest predictive and/or classification variables(s) for accurately identifying skin spots or related spot segments (e.g., 202azmap1) within the spot map (e.g., 202azmap). Still further, a neural network may have been trained with spot maps in order to configured one or more nodes or hidden layers to identify a highest predictive and/or classification variable(s) for accurately identifying skin spots or related spot segments (e.g., 202azmap1) within the spot map (e.g., 202azmap).

In another embodiment, the RGB image given in FIG. 4C is converted into hemoglobin and melanin images (FIGS. 4D and 4E respectively) using a regression model, a machine learning model (such as independent component analysis, principal component analysis) or a deep learning model (such as a generative neural network). Hemoglobin and melanin level are measured by overlaying spots map in predicted hemoglobin and melanin images. Spots are classified into melanin spots or hemoglobin spots based on the ratio of hemoglobin to melanin. This can be further optimized by normalizing spot melanin and hemoglobin values to the melanin and hemoglobin values of the surrounding skin.

The instructions, when executed by the processors, may further cause output, by the prediction or classification model, of a prediction or classification value indicating a spot type (e.g., a hemoglobin and/or melanin spot type). For example, step 406 demonstrates classification of a hemoglobin spot type, where FIG. 4D illustrates image 202azhem having a spot 202azhem1 identified as a hemoglobin spot type. By contrast, step 408 demonstrates classification of a melanin spot type, where FIG. 4E illustrates image 202azmel having a spot 202azmel1 identified as a melanin spot type. In some aspects, the spot type or otherwise spot classification may be identified by spot identifiers (IDs), where one set of spot IDs (e.g., IDs 1-7) are selected to identify one group of spots identifying hemoglobin spots of various levels of pigmentations or intensities of the skin as identifiable within the pixel data based on pixel values (e.g., RGB and/or L*a*b* values), and where another set of spot IDs (e.g., IDs 10-17) are selected to identify another group of spots identifying melanin spots of various levels of pigmentations or intensities of the skin as identifiable within the pixel data based on pixel values (e.g., RGB and/or L*a*b* values). It is to be understood that additional and/or different spot IDs may be used to detect or classify additional and/or different types of spots or discolorations on a user skin.

Further with respect to FIGS. 4A-4E, the computing instruction may further be executed to determine, based on the prediction or classification value (e.g., spot ID), at least one spot classification (e.g., hemoglobin type spot or melanin type spot) selected from the one or more spot classifications of the skin-based learning model. The spot type can be classified as an inflammatory (red) spot indicative of hemoglobin presence in the skin, and/or a pigmentary (brown) spot indicative of melanin presence in the skin. In some aspects, the spot type can be based on a ratio of detected hemoglobin-to-melanin presence. For example, in some aspects the prediction or classification value as output by the skin-based learning model may be used to generate or update a chromophore image, where aspects of the pixel values (e.g., indicative of hemoglobin and/or melanin) are overlayed on top of one another and the ratio of detected hemoglobin-to-melanin may be determined from the resulting pixel(s) of the chromophore image. In such aspects, the a spot classification or type may be determined based on a dominance (e.g., percentage) of one type over the other. Additionally, or alternatively, the classification may comprise a hybrid type where two or more types (e.g., both hemoglobin and melanin) are identified as present at the spot location. In such aspects, at least one spot feature identifiable within the pixel data and/or the one or more spot classification(s) may be based on biological chromophores of skin comprising one or more of: eumelanin, pheomelanin, oxyhemoglobin, deoxyhemoglobin, bilirubin, or oxidized sebum.

One or more products may be than be recommended to treat the identified spot type(s), where, for example, for a hemoglobin type spot, an anti-inflammation product may be recommended. For a melanin type spot, a product for treating hyperpigmentation may be recommended.

FIG. 5 illustrates an example user interface 502 as rendered on a display screen 500 of a user computing device (e.g., user computing device 111c1) in accordance with various embodiments disclosed herein. For example, as shown in the example of FIG. 5, user interface 502 may be implemented or rendered via an application (app) executing on user computing device 111c1. For example, as shown in the example of FIG. 5, user interface 502 may be implemented or rendered via a native app executing on user computing device 111c1. In the example of FIG. 5, user computing device 111c1 is a user computer device as described for FIG. 1, e.g., where 111c1 is illustrated as an APPLE iPhone that implements the APPLE iOS operating system and that has display screen 500. User computing device 111c1 may execute one or more native applications (apps) on its operating system, including, for example, imaging app as described herein. Such native apps may be implemented or coded (e.g., as computing instructions) in a computing language (e.g., SWIFT) executable by the user computing device operating system (e.g., APPLE iOS) by the processor of user computing device 111c1.

Additionally, or alternatively, user interface 502 may be implemented or rendered via a web interface, such as via a web browser application, e.g., Safari and/or Google Chrome app(s), or other such web browser or the like.

As shown in the example of FIG. 5, user interface 502 comprises a graphical representation (e.g., of image 202az or portion thereof) of a user's skin. Image 202az may comprise the image of the user (or graphical representation thereof) comprising pixel data (e.g., pixel data 202ap) of at least a portion of a skin region of the user's skin as described herein. In the example of FIG. 5, graphical representation (e.g., image 202az) of the user is annotated with one or more graphics (e.g., areas of pixel data 202ap) or textual rendering(s) (e.g., text 202at) corresponding to various features identifiable within the pixel data comprising a portion of a skin region of the user. For example, the area of pixel data 202ap may be annotated or overlaid on top of the image of the user (e.g., image 202az) to highlight the area or feature(s) identified within the pixel data (e.g., feature data and/or raw pixel data) by the skin-based learning model (e.g., skin-based learning model 108). In the example of FIG. 5, the area of pixel data 202ap indicates features, as defined in pixel data 202ap, indicating a melanin spot (e.g., for pixels at or near 202ap3), and may indicate other features shown in area of pixel data 202ap, as described herein. In various embodiments, the pixels identified as the specific features (e.g., any one of pixels 202ap1-3), may be highlighted or otherwise annotated when rendered on display screen 500.

Textual rendering (e.g., text 202at) shows a user-specific attribute or feature (e.g., value “14” for pixel 202ap3) which may indicate that the pixel(s) near or at pixel 202ap3 has a spot ID of 14 for coloring of the skin at that area. The 14 ID (e.g., on a scale of 11-17) indicates that the user has a mild or otherwise enhanced color anomaly compared to the user's other skin in the given skin region (e.g., of the skin region of 202az), such that the user would likely benefit from using a product to improve their skin quality and or appearance (e.g., to normalize the spot or otherwise skin discoloration). It is to be understood that other textual rendering types or values are contemplated herein, where textual rendering types or values may be rendered, for example, such as spot IDs for melanin, hemoglobin, and/or the like. Additionally, or alternatively, color values may be used and/or overlaid on a graphical representation shown on user interface 502 (e.g., image 202az) to indicate a degree or quality of a given spot ID, e.g., a high ID of 17 or a low ID of 11 (e.g., low RGB and/or L*a*b* pixel values), or otherwise. The IDs may be provided as raw values, absolute scores, percentage based, IDs. Additionally, or alternatively, such IDs may be presented with textual or graphical indicators indicating whether or not an ID is representative of positive results (e.g., low discoloration indicating low sun exposure or skin irritation), negative results (e.g., high discoloration indicating excessive sun exposure or skin irritation), or acceptable results (average or acceptable values).

User interface 502 may also include or render a user-specific spot classification 510. In the embodiment of FIG. 5, the user-specific spot classification 510 comprises a message 510m to the user designed to indicate the user-specific spot classification to the user, along with a brief description of any reasons resulting in the user-specific spot classification. As shown in the example of FIG. 5, message 512m indicates to a user that the user-specific spot classification is “mild” (e.g., level 14) and further indicates to the user that the user-specific spot classification results from hyper melanin at the indicated region of the user's skin.

User interface 502 may also include or render a user-specific skin recommendation 512. For example, the imaging app may render, on a display screen of a computing device, at least one user-specific skin recommendation based on the user-specific spot classification. In various aspects, the user-specific skin recommendation may comprise a textual recommendation, an imaged based recommendation, and/or virtual rendering of the at least the portion of the skin region of the user. For example, In the embodiment of FIG. 5, user-specific skin recommendation 512 comprises a message 512m to the user designed to address at least one feature identifiable within the pixel data comprising the portion of a skin region of the user's skin. As shown in the example of FIG. 5, message 512m recommends to the user to use a night face cream to help reduce dark spots. The night face cream product may be a composition of hydroxycinnamic acids (HCAs) and niacinamide at a low pH as described herein. The product recommendation can be made based on the spot ID (e.g., value 14) suggesting that the image of the user depicts a mild degree of discoloration, where the night cream product is designed to address discoloration detected or classified in the pixel data of image 202az or otherwise assumed based on the spot ID, or classification, as output by model 108. The product recommendation can be correlated to the identified feature within the pixel data, and the user computing device 111c1 and/or server(s) 102 can be instructed to output the product recommendation when the feature (e.g., hyper melanin) is identified or classified.

User interface 502 may also include or render a section for a specific product recommendation 522 for a manufactured product 524r (e.g., night face cream as described above). The product recommendation 522 may correspond to the user-specific skin recommendation 512, as described above. For example, in the example of FIG. 5, the user-specific skin recommendation 512 may be displayed on display screen 500 of user computing device 111c1 with instructions (e.g., message 512m) for treating, with the manufactured product (manufactured product 524r (e.g., night face cream)) at least one feature (e.g., mild spot ID of 14 related to melanin at pixels near or at 202ap3) identifiable in the pixel data (e.g., pixel data 202ap) comprising pixel data of at least a portion of a skin region of the user's skin.

As shown in FIG. 5, user interface 502 recommends a product (e.g., manufactured product 524r (e.g., night face cream)) based on the user-specific skin recommendation 512. In the example of FIG. 5, the output or analysis of image(s) (e.g. image 202az) of skin-based learning model (e.g., skin-based learning model 108), e.g., user-specific spot classification 510 and/or its related values (e.g., value 3) or related pixel data (e.g., 202ap1, 202ap2, and/or 202ap3), and/or the user-specific skin recommendation 512, may be used to generate or identify recommendations for corresponding product(s). Such recommendations may include products such as night face cream, skin exfoliants, skin moisturizers, moisturizing treatments, information about avoiding excessive sun exposure, and the like to address the user-specific issue as detected within the pixel data by the skin-based learning model (e.g., skin-based learning model 108).

In the example of FIG. 5, user interface 502 renders or provides a recommended product (e.g., manufactured product 524r) as determined by skin-based learning model (e.g., skin-based learning model 108) and its related image analysis of image 202az and its pixel data and various features. In the example of FIG. 5, this is indicated and annotated (524p) on user interface 502.

User interface 502 may further include a selectable UI button 524s to allow the user (e.g., the user of image 202az) to select for purchase or shipment the corresponding product (e.g., manufactured product 524r). In some embodiments, selection of selectable UI button 524s may cause the recommended product(s) to be shipped to the user (e.g., user of image 202a) and/or may notify a third party that the individual is interested in the product(s). For example, either user computing device 111c1 and/or imaging server(s) 102 may initiate, based on the user-specific spot classification 510 and/or the user-specific skin recommendation 512, the manufactured product 524r (e.g., night face cream) for shipment to the user. In such embodiments, the product may be packaged and shipped to the user.

In various embodiments, a graphical representation (e.g., image 202az), with graphical annotations (e.g., area of pixel data 202ap), textual annotations (e.g., text 202at), and the user-specific spot classification 510 and the user-specific skin recommendation 512 may be transmitted, via the computer network (e.g., from an imaging server 102 and/or one or more processors) to user computing device 111c1, for rendering on display screen 500. In other embodiments, no transmission to the imaging server of the user's specific image occurs, where the user-specific spot classification 510 and the user-specific skin recommendation 512 (and/or product specific recommendation) may instead be generated locally, by the skin-based learning model (e.g., skin-based learning model 108) executing and/or implemented on the user's mobile device (e.g., user computing device 111c1) and rendered, by a processor of the mobile device, on display screen 500 of the mobile device (e.g., user computing device 111c1).

In some embodiments, any one or more of graphical representations (e.g., image 202az), with graphical annotations (e.g., area of pixel data 202ap), textual annotations (e.g., text 202at), user-specific spot classification 510, user-specific skin recommendation 512, and/or product recommendation 522 may be rendered (e.g., rendered locally on display screen 500) in real-time or near-real time during or after receiving, the image having the skin region of the user's skin. In embodiments where the image is analyzed by imaging server(s) 102, the image may be transmitted and analyzed in real-time or near real-time by imaging server(s) 102.

In some embodiments, the user may provide a new image that may be transmitted to imaging server(s) 102 for updating, retraining, or reanalyzing by skin-based learning model 108. In other embodiments, a new image that may be locally received on computing device 111c1 and analyzed, by skin-based learning model 108, on the computing device 111c1.

In addition, as shown in the example of FIG. 5, the user may select selectable button 512i for reanalyzing (e.g., either locally at computing device 111c1 or remotely at imaging server(s) 102) a new image. Selectable button 512i may cause user interface 502 to prompt the user to attach for analyzing a new image. Imaging server(s) 102 and/or a user computing device such as user computing device 111c1 may receive a new image comprising pixel data of at least a portion of a skin region of the user's skin. The new image may be captured by the imaging device. The new image (e.g., similar to image 202az) may comprise pixel data of at least a portion of a skin region of the user's skin. The skin-based learning model (e.g., skin-based learning model 108), executing on the memory of the computing device (e.g., imaging server(s) 102), may analyze the new image captured by the imaging device to determine an image classification of the user's skin region. The computing device (e.g., imaging server(s) 102) may generate, based on a comparison of the image and the second image or the classification and the second classification of the user's skin region, a new user-specific spot classification and/or a new user-specific skin recommendation regarding at least one feature identifiable within the pixel data of the new image. For example, the new user-specific spot classification may include a new graphical representation including graphics and/or text (e.g., showing a new skin spot ID value, e.g., 11, after the user used a night face cream). The new user-specific spot classification may include additional spot classifications, e.g., that the user has successfully used night face cream to reduce melanin as detected with the pixel data of the new image. A comment may include that the user needs to correct additional features detected within the pixel data, e.g., any additional spots by applying an additional product, e.g., moisturizing oil or the like.

In various embodiments, the new user-specific spot classification and/or the new user-specific skin recommendation may be transmitted via the computer network, from server(s) 102, to the user computing device of the user for rendering on the display screen 500 of the user computing device (e.g., user computing device 111c1).

In other embodiments, no transmission to the imaging server of the user's new image occurs, where the new user-specific spot classification and/or the new user-specific skin recommendation (and/or product specific recommendation) may instead be generated locally, by the skin-based learning model (e.g., skin-based learning model 108a) executing and/or implemented on the user's mobile device (e.g., user computing device 111c1) and rendered, by a processor of the mobile device, on a display screen of the mobile device (e.g., user computing device 111c1).

### Aspects of the Disclosure

The following aspects are provided as examples in accordance with the disclosure herein and are not intended to limit the scope of the disclosure.

1. A digital imaging and artificial intelligence-based system configured to analyze pixel data of an image of user skin to generate one or more user-specific skin spot classifications, the digital imaging and artificial intelligence-based system comprising: one or more processors; an imaging application (app) comprising computing instructions configured to execute on the one or more processors; and a skin-based learning model, accessible by the imaging app, and trained with pixel data of a plurality of training images depicting skin of respective individuals, the skin-based learning model configured to output one or more spot classifications corresponding to one or more spot features of skin regions of the respective individuals, wherein the computing instructions of the imaging app when executed by the one or more processors, cause the one or more processors to: receive an image of a user, the image comprising a digital image as captured by an imaging device, and the image comprising pixel data of at least a portion of a skin region of the user, analyze, by the skin-based learning model, the image as captured by the imaging device to determine at least one spot classification of the user's skin, the at least one spot classification selected from the one or more spot classifications of the skin-based learning model, and generate, based on the at least one spot classification of the user's skin, a user-specific skin recommendation designed to address at least one spot feature identifiable within the pixel data comprising the portion of the skin region of the user.

2. The digital imaging and artificial intelligence-based system of aspect 1, wherein the at least one spot feature identifiable within the pixel data or the one or more spot classifications is based on biological chromophores of skin comprising one or more of: eumelanin, pheomelanin, oxyhemoglobin, deoxyhemoglobin, bilirubin, or oxidized sebum.

3. The digital imaging and artificial intelligence-based system of any one or more of aspects 1-2, wherein the one or more spot classifications comprise one or more of: (1) a hemoglobin type classification; or (2) a melanin type classification.

4. The digital imaging and artificial intelligence-based system of any one or more of aspects 1-3, wherein an image calibration algorithm is applied to each of the plurality of training images to alter the images to enhance spot classification, and wherein the computing instructions of the imaging app when executed by the one or more processors, further cause the one or more processors to: apply the image calibration algorithm to the image of the user prior to analyzing, with the skin-based learning model, the image of the user.

5. The digital imaging and artificial intelligence-based system of any one or more of aspects 1-3, wherein the skin-based learning model is an ensemble-based AI model comprising (i) a segmentation model configured to generate a segmentation mapping of one or more spots in a skin region of an image, and (ii) a prediction or classification model configured to analyze the pixel data of the segmentation mapping of one or more spots, and wherein the computing instructions of the imaging app when executed by the one or more processors, further cause the one or more processors to: generate a user-specific segmentation mapping of one or more spots in the portion of the skin region of the user identifiable in the image of the user; output, by the prediction or classification model, a prediction or classification value indicating a spot type; and determine, based on the prediction or classification value, the at least one spot classification selected from the one or more spot classifications of the skin-based learning model.

6. The digital imaging and artificial intelligence-based system of any one or more of aspects 1-4, wherein each image of the one or more of the plurality of training images or the image of the user comprises at least one cropped image depicting the skin region having a single instance of a spot feature.

7. The digital imaging and artificial intelligence-based system of any one or more of aspects 1-5, wherein each image of the one or more of the plurality of training images or the image of the user comprises multiple angles or perspectives depicting skin regions of the respective individuals or the user.

8. The digital imaging and artificial intelligence-based system of any one or more of aspects 1-6, wherein the computing instructions of the imaging app when executed by the one or more processors, further cause the one or more processors to: render, on a display screen of a computing device, at least one user-specific skin recommendation based on the user-specific spot classification.

9. The digital imaging and artificial intelligence-based system of any one or more of aspects 1-7, wherein the at least one user-specific skin recommendation is displayed on the display screen of the computing device with instructions for treating the at least one spot feature identifiable in the pixel data comprising the portion of the skin region of the user.

10. The digital imaging and artificial intelligence-based system of any one or more of aspects 1-8, wherein the at least one user-specific skin recommendation comprises a textual recommendation, an imaged based recommendation, or virtual rendering of the at least the portion of the skin region of the user.

11. The digital imaging and artificial intelligence-based system of any one or more of aspects 1-9, wherein the at least one user-specific skin recommendation is rendered on the display screen in real-time or near-real time, during, or after receiving, the image of the user.

12. The digital imaging and artificial intelligence-based system of aspect 7 wherein the at least one user-specific spot recommendation comprises a product recommendation for a manufactured product.

13. The digital imaging and artificial intelligence-based system of aspect 11, wherein the at least one user-specific skin recommendation is displayed on the display screen of the computing device with instructions for treating, with the manufactured product, the at least one spot feature identifiable in the pixel data comprising the portion of a skin region of the user.

14. The digital imaging and artificial intelligence-based system of aspect 11, wherein the computing instructions further cause the one or more processors to: initiate, based on the at least one user-specific skin recommendation, the manufactured product for shipment to the user.

15. The digital imaging and artificial intelligence-based system of aspect 11, wherein the computing instructions further cause the one or more processors to: generate a modified image based on the image, the modified image depicting how the user's skin region is predicted to appear after treating the at least one spot feature with the manufactured product; and render, on the display screen of the computing device, the modified image.

16. The digital imaging and artificial intelligence-based system of any one or more of aspects 1-14, wherein the computing instructions of the imaging app when executed by the one or more processors, further cause the one or more processors to: generate a skin quality code as determined based on the user-specific spot classification designed to address the at least one spot feature identifiable within the pixel data comprising the portion of the skin region of the user.

17. The digital imaging and artificial intelligence-based system of any one or more of aspects 1-15, wherein the computing instructions further cause the one or more processors to: record, in one or more memories communicatively coupled to the one or more processors, the image of the user as captured by the imaging device at a first time for tracking changes to user's skin region over time, receive a second image of the user, the second image captured by the imaging device at a second time, and the second image comprising pixel data of at least a portion of a skin region of the user, analyze, by the skin-based learning model, the second image captured by the imaging device to determine, at the second time, a second image classification of the user's skin region as selected from the one or more image classifications of the skin-based learning model, and generate, based on a comparison of the image and the second image and/or the image classification and the second image classification of the user's skin region, a new user-specific spot classification regarding at least one spot feature identifiable or lack thereof within the pixel data of the second image comprising at least the portion the skin region of the user.

18. The digital imaging and artificial intelligence-based system of any one or more of aspects 1-16, wherein the skin-based learning model is an artificial intelligence (AI) based model trained with at least one AI algorithm.

19. The digital imaging and artificial intelligence-based system of any one or more of aspects 1-17, wherein the one or more spot features of skin regions of the plurality of training images differ based one or more user demographics or ethnicities of the respective individuals, and wherein the user-specific spot classification of the user is generated, by the skin-based learning model, based on an ethnicity or demographic value of the user.

20. The digital imaging and artificial intelligence-based system of any one or more of aspects 1-19, wherein the skin-based learning model is further trained with user demographic data and environment data of the respective users, and wherein the at least one spot classification, as generated by the skin-based learning model is further based on user demographic data and environment data as provided by the user.

21. The digital imaging and artificial intelligence-based system of any one or more of aspects 1-20, wherein at least one of the one or more processors comprises a processor of a mobile device, and wherein the imaging device comprises a digital camera of the mobile device.

22. The digital imaging and artificial intelligence-based system of any one or more of aspects 1-21, wherein the one or more processors comprises a server processor of a server, wherein the server is communicatively coupled to a computing device via a computer network, and where the imaging app comprises a server app portion configured to execute on the one or more processors of the server and a computing device app portion configured to execute on one or more processors of the computing device, the server app portion configured to communicate with the computing device app portion, wherein the server app portion is configured to implement one or more of: (1) receiving the image captured by the imaging device; (2); determining the at least one spot classification of the user's skin region; (3) generating the user-specific spot classification; and/or (4) transmitting a user-specific recommendation the computing device app portion.

23. A digital imaging and artificial intelligence-based method for analyzing pixel data of an image of user skin to generate one or more user-specific skin spot classifications, the digital imaging and artificial intelligence-based method comprising: receiving, at one or more processors, an image of a user, the image comprising a digital image as captured by an imaging device, and the image comprising pixel data of at least a portion of a skin region of the user; analyzing, by a skin-based learning model executing on the one or more processors, the image as captured by the imaging device to determine at least one spot classification of the user's skin, the at least one spot classification selected from the one or more spot classifications of the skin-based learning model, wherein the skin-based learning model has been trained with pixel data of a plurality of training images depicting skin of respective individuals, the skin-based learning model configured to output one or more spot classifications corresponding to one or more spot features of skin regions of the respective individuals; and generating, by the one or more processors and based on the at least one spot classification of the user's skin, a user-specific skin recommendation designed to address at least one spot feature identifiable within the pixel data comprising the portion of the skin region of the user.

24. The digital imaging and artificial intelligence-based method of aspect 23, wherein the at least one spot feature identifiable within the pixel data or the one or more spot classifications is based on biological chromophores of skin comprising one or more of: eumelanin, pheomelanin, oxyhemoglobin, deoxyhemoglobin, bilirubin, or oxidized sebum.

25. The digital imaging and artificial intelligence-based method of any one or more of aspects 23-24, wherein the one or more spot classifications comprise one or more of: (1) a hemoglobin type classification; or (2) a melanin type classification.

26. The digital imaging and artificial intelligence-based method of any one or more of aspects 23-25, wherein an image calibration algorithm is applied to each of the plurality of training images to alter the images to enhance spot classification, and wherein the computing instructions of the imaging app when executed by the one or more processors, further cause the one or more processors to: apply the image calibration algorithm to the image of the user prior to analyzing, with the skin-based learning model, the image of the user.

27. The digital imaging and artificial intelligence-based method of any one or more of aspects 23-26, wherein the skin-based learning model is an ensemble-based AI model comprising (i) a segmentation model configured to generate a segmentation mapping of one or more spots in a skin region of an image, and (ii) a prediction or classification model configured to analyze the pixel data of the segmentation mapping of one or more spots, and wherein the computing instructions of the imaging app when executed by the one or more processors, further cause the one or more processors to: generate a user-specific segmentation mapping of one or more spots in the portion of the skin region of the user identifiable in the image of the user; output, by the prediction or classification model, a prediction or classification value indicating a spot type; and determine, based on the prediction or classification value, the at least one spot classification selected from the one or more spot classifications of the skin-based learning model.

28. The digital imaging and artificial intelligence-based method of any one or more of aspects 23-27, wherein each image of the one or more of the plurality of training images or the image of the user comprises at least one cropped image depicting the skin region having a single instance of a spot feature.

29. The digital imaging and artificial intelligence-based method of any one or more of aspects 23-28, wherein each image of the one or more of the plurality of training images or the image of the user comprises multiple angles or perspectives depicting skin regions of the respective individuals or the user.

30. The digital imaging and artificial intelligence-based method of any one or more of aspects 23-29, wherein the computing instructions of the imaging app when executed by the one or more processors, further cause the one or more processors to: render, on a display screen of a computing device, at least one user-specific skin recommendation based on the user-specific spot classification.

31. The digital imaging and artificial intelligence-based method of aspect 30, wherein the at least one user-specific skin recommendation is displayed on the display screen of the computing device with instructions for treating the at least one spot feature identifiable in the pixel data comprising the portion of the skin region of the user.

32. The digital imaging and artificial intelligence-based method of aspect 30, wherein the at least one user-specific skin recommendation comprises a textual recommendation, an imaged based recommendation, or virtual rendering of the at least the portion of the skin region of the user.

33. The digital imaging and artificial intelligence-based method of aspect 30, wherein the at least one user-specific skin recommendation is rendered on the display screen in real-time or near-real time, during, or after receiving, the image of the user.

34. The digital imaging and artificial intelligence-based method of aspect 30 wherein the at least one user-specific spot recommendation comprises a product recommendation for a manufactured product.

35. The digital imaging and artificial intelligence-based method of aspect 34, wherein the at least one user-specific skin recommendation is displayed on the display screen of the computing device with instructions for treating, with the manufactured product, the at least one spot feature identifiable in the pixel data comprising the portion of a skin region of the user.

36. The digital imaging and artificial intelligence-based method of aspect 34, wherein the computing instructions further cause the one or more processors to: initiate, based on the at least one user-specific skin recommendation, the manufactured product for shipment to the user.

37. The digital imaging and artificial intelligence-based method of aspect 34, wherein the computing instructions further cause the one or more processors to: generate a modified image based on the image, the modified image depicting how the user's skin region is predicted to appear after treating the at least one spot feature with the manufactured product; and render, on the display screen of the computing device, the modified image.

38. The digital imaging and artificial intelligence-based method of any one or more of aspects 23-37, wherein the computing instructions of the imaging app when executed by the one or more processors, further cause the one or more processors to: generate a skin quality code as determined based on the user-specific spot classification designed to address the at least one spot feature identifiable within the pixel data comprising the portion of the skin region of the user.

39. The digital imaging and artificial intelligence-based method of any one or more of aspects 23-38, wherein the computing instructions further cause the one or more processors to: record, in one or more memories communicatively coupled to the one or more processors, the image of the user as captured by the imaging device at a first time for tracking changes to user's skin region over time, receive a second image of the user, the second image captured by the imaging device at a second time, and the second image comprising pixel data of at least a portion of a skin region of the user, analyze, by the skin-based learning model, the second image captured by the imaging device to determine, at the second time, a second image classification of the user's skin region as selected from the one or more image classifications of the skin-based learning model, and generate, based on a comparison of the image and the second image and/or the image classification and the second image classification of the user's skin region, a new user-specific spot classification regarding at least one spot feature identifiable or lack thereof within the pixel data of the second image comprising at least the portion the skin region of the user.

40. The digital imaging and artificial intelligence-based method of any one or more of aspects 23-39, wherein the skin-based learning model is an artificial intelligence (AI) based model trained with at least one AI algorithm.

41. The digital imaging and artificial intelligence-based method of aspect 40, wherein the one or more spot features of skin regions of the plurality of training images differ based one or more user demographics or ethnicities of the respective individuals, and wherein the user-specific spot classification of the user is generated, by the skin-based learning model, based on an ethnicity or demographic value of the user.

42. The digital imaging and artificial intelligence-based method of any one or more of aspects 23-21, wherein the skin-based learning model is further trained with user demographic data and environment data of the respective users, and wherein the at least one spot classification, as generated by the skin-based learning model is further based on user demographic data and environment data as provided by the user.

43. The digital imaging and artificial intelligence-based method of any one or more of aspects 23-41, wherein at least one of the one or more processors comprises a processor of a mobile device, and wherein the imaging device comprises a digital camera of the mobile device.

44. The digital imaging and artificial intelligence-based method of any one or more of aspects 23-43, wherein the one or more processors comprises a server processor of a server, wherein the server is communicatively coupled to a computing device via a computer network, and where the imaging app comprises a server app portion configured to execute on the one or more processors of the server and a computing device app portion configured to execute on one or more processors of the computing device, the server app portion configured to communicate with the computing device app portion, wherein the server app portion is configured to implement one or more of: (1) receiving the image captured by the imaging device; (2); determining the at least one spot classification of the user's skin region; (3) generating the user-specific spot classification; and/or (4) transmitting a user-specific recommendation the computing device app portion.

45. A tangible, non-transitory computer-readable medium storing instructions for analyzing pixel data of an image of user skin to generate one or more user-specific skin spot classifications, that when executed by one or more processors cause the one or more processors to: receive an image of a user, the image comprising a digital image as captured by an imaging device, and the image comprising pixel data of at least a portion of a skin region of the user; analyze, by a skin-based learning model, the image as captured by the imaging device to determine at least one spot classification of the user's skin, the at least one spot classification selected from the one or more spot classifications of the skin-based learning model, wherein the skin-based learning model has been trained with pixel data of a plurality of training images depicting skin of respective individuals, the skin-based learning model configured to output one or more spot classifications corresponding to one or more spot features of skin regions of the respective individuals; and generate, based on the at least one spot classification of the user's skin, a user-specific skin recommendation designed to address at least one spot feature identifiable within the pixel data comprising the portion of the skin region of the user.

### ADDITIONAL CONSIDERATIONS

Although the disclosure herein sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location, while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other embodiments, the one or more processors or processor-implemented modules may be distributed across a number of geographic locations.

This detailed description is to be construed as exemplary only and does not describe every possible embodiment, as describing every possible embodiment would be impractical, if not impossible. A person of ordinary skill in the art may implement numerous alternate embodiments, using either current technology or technology developed after the filing date of this application.

Those of ordinary skill in the art will recognize that a wide variety of modifications, alterations, and combinations can be made with respect to the above described embodiments without departing from the scope of the invention, and that such modifications, alterations, and combinations are to be viewed as being within the ambit of the inventive concept.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A digital imaging and artificial intelligence-based system configured to analyze pixel data of an image of user skin to generate one or more user-specific skin spot classifications, the digital imaging and artificial intelligence-based system comprising:
one or more processors;
an imaging application (app) comprising computing instructions configured to execute on the one or more processors; and
a skin-based learning model, accessible by the imaging app, and trained with pixel data of a plurality of training images depicting skin of respective individuals, the skin-based learning model configured to output one or more spot classifications corresponding to one or more spot features of skin regions of the respective individuals,
wherein the computing instructions of the imaging app when executed by the one or more processors, cause the one or more processors to:
receive an image of a user, the image comprising a digital image as captured by an imaging device, and the image comprising pixel data of at least a portion of a skin region of the user,
analyze, by the skin-based learning model, the image as captured by the imaging device to determine at least one spot classification of the user's skin, the at least one spot classification selected from the one or more spot classifications of the skin-based learning model, and
generate, based on the at least one spot classification of the user's skin, a user-specific skin recommendation designed to address at least one spot feature identifiable within the pixel data comprising the portion of the skin region of the user.

2. The digital imaging and artificial intelligence-based system of claim 1, wherein the at least one spot feature identifiable within the pixel data or the one or more spot classifications is based on biological chromophores of skin comprising one or more of: eumelanin, pheomelanin, oxyhemoglobin, deoxyhemoglobin, bilirubin, or oxidized sebum.

3. The digital imaging and artificial intelligence-based system of claim 1, wherein the one or more spot classifications comprise one or more of: (1) a hemoglobin type classification; or (2) a melanin type classification.

4. The digital imaging and artificial intelligence-based system of claim 1, wherein an image calibration algorithm is applied to each of the plurality of training images to alter the images to enhance spot classification, and
wherein the computing instructions of the imaging app when executed by the one or more processors, further cause the one or more processors to:
apply the image calibration algorithm to the image of the user prior to analyzing, with the skin-based learning model, the image of the user.

5. The digital imaging and artificial intelligence-based system of claim 1, wherein the skin-based learning model is an ensemble-based AI model comprising (i) a segmentation model configured to generate a segmentation mapping of one or more spots in a skin region of an image, and (ii) a prediction or classification model configured to analyze the pixel data of the segmentation mapping of one or more spots, and
wherein the computing instructions of the imaging app when executed by the one or more processors, further cause the one or more processors to:
generate a user-specific segmentation mapping of one or more spots in the portion of the skin region of the user identifiable in the image of the user,
output, by the prediction or classification model, a prediction or classification value indicating a spot type, and
determine, based on the prediction or classification value, the at least one spot classification selected from the one or more spot classifications of the skin-based learning model.

6. The digital imaging and artificial intelligence-based system of claim 1, wherein each image of the one or more of the plurality of training images or the image of the user comprises at least one cropped image depicting the skin region having a single instance of a spot feature.

7. The digital imaging and artificial intelligence-based system of claim 1, wherein each image of the one or more of the plurality of training images or the image of the user comprises multiple angles or perspectives depicting skin regions of the respective individuals or the user.

8. The digital imaging and artificial intelligence-based system of claim 1, wherein the computing instructions of the imaging app when executed by the one or more processors, further cause the one or more processors to:
render, on a display screen of a computing device, at least one user-specific skin recommendation based on the user-specific spot classification.

9. The digital imaging and artificial intelligence-based system of claim 8, wherein the at least one user-specific skin recommendation is displayed on the display screen of the computing device with instructions for treating the at least one spot feature identifiable in the pixel data comprising the portion of the skin region of the user.

10. The digital imaging and artificial intelligence-based system of claim 8, wherein the at least one user-specific skin recommendation comprises a textual recommendation, an imaged based recommendation, or virtual rendering of the at least the portion of the skin region of the user.

11. The digital imaging and artificial intelligence-based system of claim 8, wherein the at least one user-specific skin recommendation is rendered on the display screen in real-time or near-real time, during, or after receiving, the image of the user.

12. The digital imaging and artificial intelligence-based system of claim 8 wherein the at least one user-specific spot recommendation comprises a product recommendation for a manufactured product.

13. The digital imaging and artificial intelligence-based system of claim 12, wherein the at least one user-specific skin recommendation is displayed on the display screen of the computing device with instructions for treating, with the manufactured product, the at least one spot feature identifiable in the pixel data comprising the portion of a skin region of the user.

14. The digital imaging and artificial intelligence-based system of claim 12, wherein the computing instructions further cause the one or more processors to:
initiate, based on the at least one user-specific skin recommendation, the manufactured product for shipment to the user.

15. The digital imaging and artificial intelligence-based system of claim 12, wherein the computing instructions further cause the one or more processors to:
generate a modified image based on the image, the modified image depicting how the user's skin region is predicted to appear after treating the at least one spot feature with the manufactured product; and
render, on the display screen of the computing device, the modified image.

16. The digital imaging and artificial intelligence-based system of claim 1, wherein the computing instructions of the imaging app when executed by the one or more processors, further cause the one or more processors to:
generate a skin quality code as determined based on the user-specific spot classification designed to address the at least one spot feature identifiable within the pixel data comprising the portion of the skin region of the user.

17. The digital imaging and artificial intelligence-based system of claim 1, wherein the computing instructions further cause the one or more processors to:
record, in one or more memories communicatively coupled to the one or more processors, the image of the user as captured by the imaging device at a first time for tracking changes to user's skin region over time,
receive a second image of the user, the second image captured by the imaging device at a second time, and the second image comprising pixel data of at least a portion of a skin region of the user,
analyze, by the skin-based learning model, the second image captured by the imaging device to determine, at the second time, a second image classification of the user's skin region as selected from the one or more image classifications of the skin-based learning model, and
generate, based on a comparison of the image and the second image and/or the image classification and the second image classification of the user's skin region, a new user-specific spot classification regarding at least one spot feature identifiable or lack thereof within the pixel data of the second image comprising at least the portion the skin region of the user.

18. The digital imaging and artificial intelligence-based system of claim 1, wherein the skin-based learning model is an artificial intelligence (AI) based model trained with at least one AI algorithm.

19. The digital imaging and artificial intelligence-based system of claim 18,
wherein the one or more spot features of skin regions of the plurality of training images differ based one or more user demographics or ethnicities of the respective individuals, and
wherein the user-specific spot classification of the user is generated, by the skin-based learning model, based on an ethnicity or demographic value of the user.

20. The digital imaging and artificial intelligence-based system of claim 1,
wherein the skin-based learning model is further trained with user demographic data and environment data of the respective users, and
wherein the at least one spot classification, as generated by the skin-based learning model is further based on user demographic data and environment data as provided by the user.

21. The digital imaging and artificial intelligence-based system of claim 1, wherein at least one of the one or more processors comprises a processor of a mobile device, and wherein the imaging device comprises a digital camera of the mobile device.

22. The digital imaging and artificial intelligence-based system of claim 1, wherein the one or more processors comprises a server processor of a server, wherein the server is communicatively coupled to a computing device via a computer network, and where the imaging app comprises a server app portion configured to execute on the one or more processors of the server and a computing device app portion configured to execute on one or more processors of the computing device, the server app portion configured to communicate with the computing device app portion, wherein the server app portion is configured to implement one or more of: (1) receiving the image captured by the imaging device; (2); determining the at least one spot classification of the user's skin region; (3) generating the user-specific spot classification; and/or (4) transmitting a user-specific recommendation the computing device app portion.

23. A digital imaging and artificial intelligence-based method for analyzing pixel data of an image of user skin to generate one or more user-specific skin spot classifications, the digital imaging and artificial intelligence-based method comprising:
receiving, at one or more processors, an image of a user, the image comprising a digital image as captured by an imaging device, and the image comprising pixel data of at least a portion of a skin region of the user;
analyzing, by a skin-based learning model executing on the one or more processors, the image as captured by the imaging device to determine at least one spot classification of the user's skin, the at least one spot classification selected from the one or more spot classifications of the skin-based learning model;
wherein the skin-based learning model has been trained with pixel data of a plurality of training images depicting skin of respective individuals, the skin-based learning model configured to output one or more spot classifications corresponding to one or more spot features of skin regions of the respective individuals, and
generating by the one or more processors and based on the at least one spot classification of the user's skin, a user-specific skin recommendation designed to address at least one spot feature identifiable within the pixel data comprising the portion of the skin region of the user.

24. A tangible, non-transitory computer-readable medium storing instructions for analyzing pixel data of an image of user skin to generate one or more user-specific skin spot classifications, that when executed by one or more processors cause the one or more processors to:
receive an image of a user, the image comprising a digital image as captured by an imaging device, and the image comprising pixel data of at least a portion of a skin region of the user;
analyze, by a skin-based learning model, the image as captured by the imaging device to determine at least one spot classification of the user's skin, the at least one spot classification selected from the one or more spot classifications of the skin-based learning model,
wherein the skin-based learning model has been trained with pixel data of a plurality of training images depicting skin of respective individuals, the skin-based learning model configured to output one or more spot classifications corresponding to one or more spot features of skin regions of the respective individuals; and
generate, based on the at least one spot classification of the user's skin, a user-specific skin recommendation designed to address at least one spot feature identifiable within the pixel data comprising the portion of the skin region of the user.
